(19) United States
(12) Patent Application Publication
(10) Pub. No.: US 2026/0126428 A1
(43) Pub. Date: May 7, 2026

(54) METHODS AND SYSTEMS TO SIMULTANEOUSLY MONITOR CANCER CELL VIABILITY AND COLLECTIVE MIGRATION IN THREE DIMENSIONS

(71) Applicants: Fred Hutchinson Cancer Center, Seattle, WA (US); University of Washington, Seattle, WA (US)

(72) Inventors: Christopher P. Miller, Seattle, WA (US); Megan Fung, Bellevue, WA (US); Scott S. Tykodi, Bellevue, WA (US); Edus H. Warren, Bainbridge Island, WA (US); Shreeram Akilesh, Seattle, WA (US)

(73) Assignees: Fred Hutchinson Cancer Center, Seattle, WA (US); University of Washington, Seattle, WA (US)

(21) Appl. No.: 19/379,406

(22) Filed: Nov. 4, 2025

Related U.S. Application Data
(60) Provisional application No. 63/716,090, filed on Nov. 4, 2024.

(51) Int. Cl. G01N 33/50; C12M 1/00; C12M 1/12; C12M 1/34; C12M 3/00; C12M 3/06

(52) U.S. Cl. CPC G01N 33/5011; C12M 21/08; C12M 23/16; C12M 25/02; C12M 29/06; C12M 41/46; G01N 33/5029; G01N 33/5082

(57) ABSTRACT

A rapid and economical human microphysiological system (“spheroid-on-a-chip”) to investigate therapies targeting collective cancer cell migration in a 3D collagen extracellular matrix is described. The 3D system can be used to identify compounds that inhibit spheroid collective migration without inducing cell death and can accelerate the development of new drugs that target metastasis.

### CROSS-REFERENCE TO RELATED APPLICATION

This application claims priority to U.S. Provisional Patent Application No. 63/716,090 filed on Nov. 4, 2024 the entire contents of which are incorporated by reference herein.

### STATEMENT OF GOVERNMENT SUPPORT

This invention was made with government support under W81XWH-19-1-0789 awarded by the Defense Health Agency, Medical Research and Development Branch. The government has certain rights in the invention.

### FIELD OF THE DISCLOSURE

The present disclosure provides methods and systems to simultaneously monitor cancer cell viability and migration in three dimensions (3D). The methods and systems can use microfluidic tissue culture-chip assays to monitor 3D collective cell migration and cell death. The disclosed platform can be used to simultaneously evaluate tumor spheroid cell death and collective migration and has utility for evaluating anti-migratory compounds.

### BACKGROUND OF THE DISCLOSURE

Cancer is the second leading cause of death in the US (Siegel et al., Cancer J Clin. 2023; 73(1):17-48) and new therapies are urgently needed. Unfortunately, the failure rate for the development of new cancer treatments is estimated to be >96% (Printz, Cancer. 2015; 121(10):1529-30). A key limitation to drug development is inadequate preclinical tumor models. The 2-dimensional (2D) growth of cancer cells in dishes does not faithfully recapitulate human tumor biology in vivo, while human xenograft and genetically engineered mouse models are expensive, time-consuming, and also fail to accurately mimic immune interactions relevant to human tumors. While immunocompetent transgenic mouse models with rapidly progressing, metastatic cancer suitable for therapeutic evaluation are available for some cancer types such as breast and prostate, they are lacking for others such as renal cell carcinoma (RCC), the most common type of kidney cancer (van der Min et al., Sci Rep. 2023; 13(1):8246). The search for more robust in vitro tumor models has stimulated interest in economical human microphysiological systems to better predict efficacy and toxicity (Ewart et al., Commun Med (Lond). 2022; 2(1):154). A key component of these systems is the ability to culture tumor cells as spheroids in a 3-dimensional (3D) extracellular matrix (ECM) which better reflects the properties of tumors in the tumor microenvironment (TME) in vivo. Advantages of 3D culture of spheroids include the ability to reproducibly define their composition and the density, stiffness, and porosity of the ECM. These systems can recapitulate in vivo tumor architecture, cell cycle heterogeneity, tumor cell-to-cell and cell-to-ECM interactions, ECM mechanical forces, central hypoxia, nutrient-, pH-, and soluble factor-gradients, gene expression, and drug resistance (Manduca et al., Front Immunol. 2023; 14:1175503; Tan et al., Adv Drug Deliv Rev. 2021; 176:113852). Importantly, 3D tumor-on-a-chip models allow for isolation and study of the impact of individual variables. In turn, this enables a stepwise increase in complexity of the model system including incorporation of the tumor vasculature, stromal and immune cells, and modulation of flow-based perfusion and soluble factor gradients.

### SUMMARY OF THE DISCLOSURE

The present disclosure provides methods and systems to simultaneously monitor cancer cell viability and migration in three dimensions (3D). The methods and systems can use microfluidic tissue culture-chip assays to monitor 3D collective cell migration and cell death. The disclosed platform can be used to simultaneously evaluate tumor spheroid cell death and collective migration and has utility for evaluating anti-migratory compounds. Spheroids are normally observed collecting at the bottom of a 3D space due to gravitational forces. The methods and systems described herein unexpectedly observed an anti-gravitational migration (i.e., away from the directional pull of gravity) of the spheroids towards the top of the 3D space. With this unexpected anti-gravitational migration, the methods and systems herein can be used to test compounds for their anti-metastatic properties (i.e., compounds with anti-metastatic properties prevent collective anti-gravitational migration of the spheroids).

### BRIEF DESCRIPTION OF THE FIGURES

The patent or application file contains at least one drawing executed in color. Copies of this patent or patent application publication with color drawing(s) will be provided by the Office upon request and payment of the necessary fee. Applicants consider the color versions of the drawings as part of the original submission and reserve the right to present color images of the drawings in later proceedings.

FIGS. 1A-1C. 3D microfluidic culture system for renal cell carcinoma (RCC) spheroids. Schematic of the idenTx 3 chip containing 3 sites per chip, each site containing injection ports for the central matrix channel and parallel media channels flanking the sites (1A, upper panel). Schematic of the structure of the posts with gaps in between on each side of the matrix channel that retains the collagen and spheroids (1A, lower panel). Common and unique significantly differentially expressed genes (log 2FC≥1, FDR<0.01) in 3D vs. 2D are shown for each cell line (1B). Mean fold increase in overlapping hypoxia genes upregulated in 3D vs. 2D in RNA-seq data for A498 and RCCC4 cells and in primary RCC vs. normal-adjacent cells in TCGA (1C).

FIGS. 2A-2C. Evaluation of bortezomib-induced cell death in 3D vs. 2D. Confocal z-stack projections of A498 spheroids (125 cells/spheroid) seeded into collagen in the chips and cultured with bortezomib (2A). The DRAQ7:CtGreen ratio for each concentration of bortezomib as the percentage of the maximum (2B). Parallel 2D cultures at concentrations of bortezomib shown as the inverse of MTS absorbance at 490 nm as a percentage of the maximum value (2C).

FIGS. 3A-3C. Collective migration of spheroids in the chip. Montage gallery views of each z-stack position of CtGreen-labeled A498 spheroids (125 cells/spheroid) immediately after seeding or after 24 hours in the presence of absence of 50 μM bortezomib (3A). Primary RCC spheroids (175 cells/spheroid) formed from tumor cells from a patient with clear cell (cc) RCC treated with or without 50 μM bortezomib for 24 hours (3B). HEPG2 spheroids (120 cells/spheroid) after 24 hours (3C).

FIGS. 4A-4C. Collective migration is an output for anti-migratory compound screening in the 3D chip. Quantification of spheroid cell death (DRAQ7/CtGreen) in confocal z-stack projections obtained at 24 hours as the fold increased vs. the untreated A498 spheroids (120 cells/spheroid) (4A). Collective migration quantified to mark the position of the spheroids for the indicated conditions (4B). Montage gallery views of the spheroid collective migration for the selected compounds (4C).

FIGS. 5A, 5B. Common differentially expressed genes in A498 and RCC4 in 3D vs. 2D. The significant common downregulated and upregulated genes in 3D A498 and RCC4 spheroids vs. 2D cultures are shown.

FIG. 6. Enriched gene expression signatures in the common differentially expressed genes in A498 and RCC4 in 3D vs. 2D. The significant (adjusted p value<0.05) gene sets from the hallmark collection in the Molecular Signatures Database enriched among the common downregulated and common upregulated genes in 3D A498 and RCC4 spheroids vs. 2D cultures are shown. Count refers to the number of overlapping genes in each gene set

### DETAILED DESCRIPTION

Cancer is the second leading cause of death in the US (Siegel et al., Cancer J Clin. 2023; 73(1):17-48) and new therapies are urgently needed. Unfortunately, the failure rate for the development of new cancer treatments is estimated to be >96% (Printz, Cancer. 2015; 121(10):1529-30). A key limitation to drug development is inadequate preclinical tumor models. The 2-dimensional (2D) growth of cancer cells in dishes does not faithfully recapitulate human tumor biology in vivo, while human xenograft and genetically engineered mouse models are expensive, time-consuming, and also fail to accurately mimic immune interactions relevant to human tumors. While immunocompetent transgenic mouse models with rapidly progressing, metastatic cancer suitable for therapeutic evaluation are available for some cancer types such as breast and prostate, they are lacking for others such as renal cell carcinoma (RCC), the most common type of kidney cancer (van der Min et al., Sci Rep. 2023; 13(1):8246). The search for more robust in vitro tumor models has stimulated interest in economical human microphysiological systems to better predict efficacy and toxicity (Ewart et al., Commun Med (Lond). 2022; 2(1):154). A key component of these systems is the ability to culture tumor cells as spheroids or organoids in a 3-dimensional (3D) extracellular matrix (ECM) which better reflects the properties of tumors in the tumor microenvironment (TME) in vivo.

The extracellular Matrix (ECM) refers to the non-cellular, three-dimensional network that surround and supports cells in tissues and in vitro cultures. The ECM is generally composed of structural proteins, such as elastin and collagens, adhesive glycoproteins, such as fibronectin and laminins, and proteoglycans/glycosaminoglycans, such as heparan sulfate, chondroitin sulfate, and hyaluronan, as well as bound water, ions, and associated signaling molecules. The ECM provides mechanical support and spatial organization, presents biochemical ligands for cell adhesion (e.g., integrin-binding motifs), and serves as a reservoir and modulator of growth factors and cytokines. The ECM's composition, density, cross-linking, and ultrastructure collectively determine tissue-level properties such as stiffness, porosity, and viscoelasticity, which in turn influences cell migration, proliferation, differentiation, and survival.

In some embodiments, “ECM” refers to both the interstitial matrix and the basement membrane. The ECM can be dynamically remodeled through synthesis, deposition, and cross-linking by resident cells, such as fibroblasts, epithelial, endothelial, or tumor cells, and through proteolysis by matrix-degrading enzymes, such as matrix metalloproteinases, and related inhibitors and/or regulators. Unless otherwise indicated, references to “ECM”, “ECM components” or “ECM-associated molecules” include native tissue matrices, decellularized matrices, solubilized or reconstituted preparations, ECM-mimetic materials such as peptide-functionalized or polymeric hydrogels presenting ECM ligands, and mixtures or fragments thereof. In some embodiments, altering the ECM composition, ligand presentation, or mechanical properties can be used to model microenvironments, to control collective or single-cell migration, or to modulate cellular responses relevant to development, wound healing, fibrosis, and cancer.

Tumor Spheroids refer to 3D, multicellular aggregates of neoplastic cells cultured ex vivo that self-assemble into approximately spherical microtissues. References to “tumor spheroids” includes single spheroids, arrays of spheroids, and pooled preparations suitable for imaging, biochemical analysis, and screening applications. The term may also include “organoid-like” spheroids derived from primary or patient-derived samples. Unless indicated otherwise, “tumor spheroids” encompass aggregates formed by non-adherent or low-attachment methods, such as hanging-drop, ultra-low-adhesion plates, microwell arrays, dynamic suspension culture methods, such as spinner flasks, orbital shakers, or matrix-assisted methods, such as embedding or overlaying in natural or synthetic hydrogels presenting extracellular-matrix ligands.

Tumor spheroids recapitulate key microenvironmental features of solid tumors, including cell-cell and cell-matrix interactions, diffusion-limited gradients of oxygen, nutrients, and metabolites, and size-dependent formation of proliferative rims, quiescent zones, and hypoxic/necrotic cores. In some embodiments, spheroids have a mean diameters of 50-1000 μm. In some embodiments spheroid with a diameter exceeding 500 μm may exhibit central hypoxia/necrosis due to mass-transport limitations. Spheroids can be generated from homogenous cancer cell lines, patient derived cells (as in the present invention), or mixed populations to model tumor-stroma interactions and therapy responses. In some embodiments, spheroids are used in high-content or high-throughput assays to evaluate proliferation, apoptosis, migration/invasion, ECM deposition, hypoxia, or drug penetration/efficacy, with readouts including diameter/volume tracking, ATP or metabolic assays, live/dead staining, immunostaining, hypoxia probes, and transcriptomic/proteomic profiling.

The number of cells per spheroid can vary. In particular embodiments, the number of cells per spheroid at least 100 cells/spheroid, at least 115 cells/spheroid, at least 120 cells/spheroid, at least 125 cells/spheroid, at least 130 cells/spheroid, at least 135 cells/spheroid, at least 140 cells/spheroid, at least 145 cells/spheroid, at least 150 cells/spheroid, at least 200 cells/spheroid, at least 250 cells/spheroid, at least 300 cells/spheroid, at least 350 cells/spheroid, at least 400 cells/spheroid, at least 450 cells/spheroid, or at least 500 cells/spheroid. The method of claim 1, wherein the tumor spheroids have 100-500 cells/spheroid. In particular embodiments, the tumor spheroids have 125 cells/spheroid. In particular embodiments, the tumor spheroids have 120 cells/spheroid.

Advantages of 3D culture of spheroids include the ability to reproducibly define their composition and the density, stiffness, and porosity of the ECM. These systems can recapitulate in vivo tumor architecture, cell cycle heterogeneity, tumor cell-to-cell and cell-to-ECM interactions, ECM mechanical forces, central hypoxia, nutrient-, pH-, and soluble factor-gradients, gene expression, and drug resistance (Manduca et al., Front Immunol. 2023; 14:1175503; Tan et al., Adv Drug Deliv Rev. 2021; 176:113852). Importantly, 3D tumor-on-a-chip models allow for isolation and study of the impact of individual variables. In turn, this enables a stepwise increase in complexity of the model system including incorporation of the tumor vasculature, stromal and immune cells, and modulation of flow-based perfusion and soluble factor gradients.

Cancer drug development has focused mainly on cytotoxic agents which often have severe side effects and has largely neglected compounds targeting cancer cell migration and metastasis, the main causes of cancer-related death.

“Cancer cell migration” refers to the coordinated process by which tumor cells leave the primary mass, traverse surrounding tissue, and access vessels en route to distant sites. Cells switch between distinct motility programs—collective (e.g., cohesive sheets/strands that retain junctions), mesenchymal (e.g., elongated, integrin-dependent, protease-assisted), and amoeboid (rounded, highly contractile, squeezing through pores with less proteolysis). These modes are plastic and can interconvert under particular environmental cues. Progression typically involves partial EMT-like changes that loosen adhesions (e.g., E-cadherin loss/redistribution), rewire the cytoskeleton, and retune integrins to latch onto specific ECM components. Cells generate traction on aligned collagen, deploy invadopodia with matrix metalloproteinases (MMPs) to breach the matrix, and follow chemotactic gradients towards vessels. Hypoxia and stromal partners reinforce motility, while matrix stiffness and LOX-mediated crosslinking guide directional migration.

“Cancer cell metastasis” refers to the cascade by which tumor cells depart the primary lesion and establish clinically relevant colonies elsewhere. It typically begins with local invasion (e.g., cells loosen junctions, remodel the ECM with MMPs, migrate through stroma), followed by intravasation into blood or lymph—often at leaky, pro-inflammatory vessels sculpted by tumor-stroma communication. In circulation, circulating tumor cells (CTCs) must endure shear stress and immune attack (e.g., platelet cloaking, DAMP/TLD signaling, checkpoint pathways). Successful cells arrest and extravasate at distant capillary beds using selectins, integrins, and proteases, then colonize the area. Most disseminated tumor cells either die or enter dormancy (e.g., cell-cycle arrest or balanced proliferation/apoptosis). Overt metastases emerge when cells adapt to—or precondition—the “pre-metastatic niche” (i.e., via tumor-secreted factors and exosomes), co-opt local stroma and vasculature, evade resident immunity, and secure metabolic support (e.g., oxidative stress buffering, organ-specific nutrients). Clinically, metastasis is tracked by endpoints like metastasis-free survival, new lesion counts, and organ-specific burden and it is the major driver of cancer mortality.

There is increasing interest in economical in vitro technologies for screening reagents that inhibit cancer cell migration and invasion and have anti-metastatic properties (Gandalovičová et al., Trends Cancer. 2017; 3(6):391-406; Solomon et al., Cells. 2021; 10(8)). “Anti-metastatic properties” refers to the capacity of a compound, biologic, device, formulation, or treatment regimen to prevent, reduce, or delay one or more steps of the metastatic cascade, including EMT, detachment from the primary tumor, degradation of extracellular matrix, invasion of surrounding stroma, intravasation, survival in the circulation, extravasation, colonization at a distant site, and/or outgrowth into macro-metastases. Anti-metastatic activity is distinct from, though it may accompany, cytostatic or cytotoxic effects on primary-tumor proliferation, and may be evidenced by attenuation of cancer-cell motility, chemotaxis, haptotaxis, invadopodia formation, matrix metalloproteinase (MMP) activity, angiogenic signaling, or pro-metastatic pathways (e.g., TGF-β, HGF/MET, PI3K/AKT, Src, Rho/ROCK). Unless indicated otherwise, “anti-metastatic properties” encompasses direct actions on tumor cells and indirect actions on stromal, endothelial, immune, or ECM components that collectively suppress dissemination and secondary-site colonization.

Current assays for anti-metastatic properties rely mainly on Boyden chambers developed in the 1960s (Boyden et al., J Exp Med. 1962; 115(3):453-66) that assess migration of 2D monolayers of cells across a porous membrane, or invasion across a minimal ECM barrier and microscopy-based approaches such as scratch assays that are laborious, low-throughput and poorly reproducible (Hulkower & Herber, Pharmaceutics. 2011; 3(1):107-24. While some assays employ chemokines to model chemoattraction, they fail to recapitulate the 3D collagen ECM which is a critical mediator of cancer cell migration and invasion in vivo (Conklin et al., Am J Pathol. 2011; 178(3):1221-32; Provenzano et al., BMC Med. 2008; 6:11).

The present disclosure provides methods and systems to monitor cancer cell viability and migration in three dimensions (3D). The methods and systems can use microfluidic tissue culture-chip assays to monitor 3D collective cell migration and cell death. The disclosed platform can be used to simultaneously evaluate tumor spheroid cell death and collective migration and has utility for evaluating anti-migratory compounds. Spheroids are normally observed collecting at the bottom of a 3D space due to gravitational forces. The methods and systems described herein unexpectedly observed an anti-gravitational migration (i.e., away from the directional pull of gravity) of the spheroids towards a top the 3D space. With this unexpected anti-gravitational migration, the methods and systems herein can be used to test compounds for their anti-metastatic properties (i.e., compounds with anti-metastatic properties prevent collective anti-gravitational migration of the spheroids).

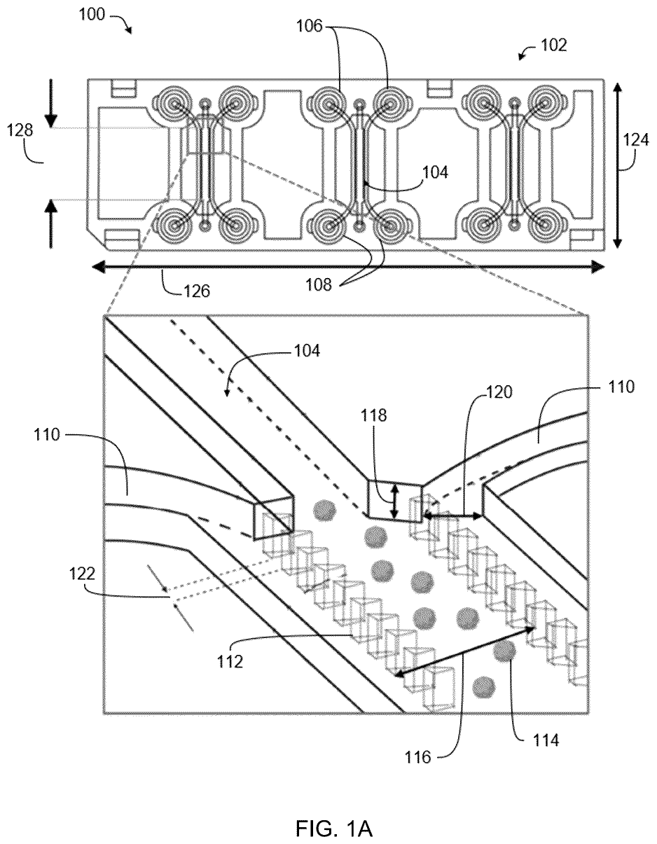

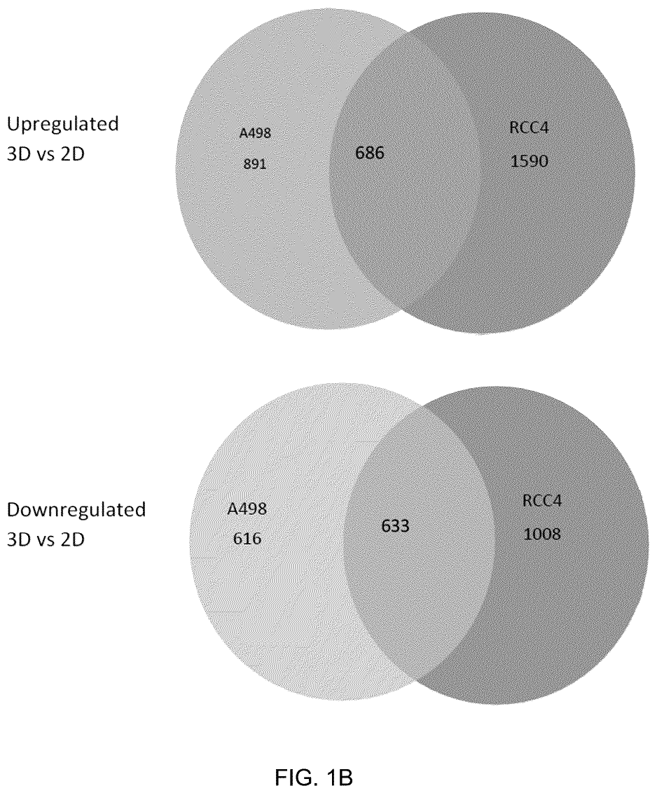

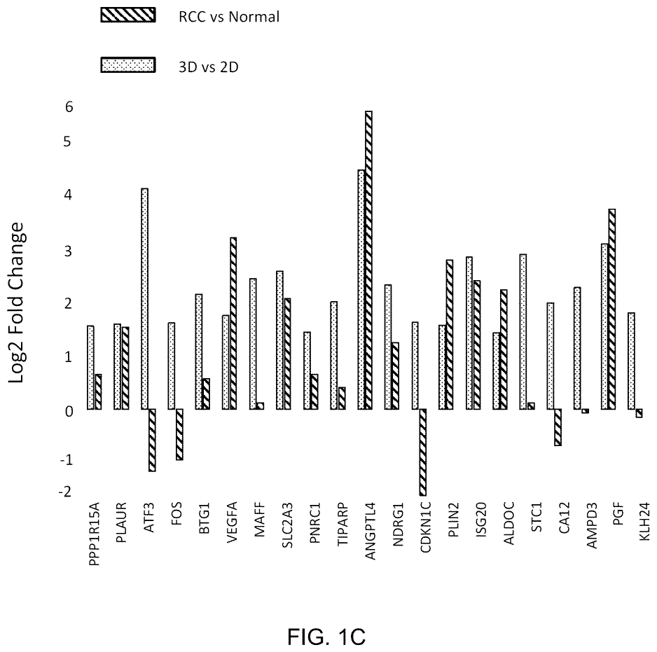

Particular embodiments can utilize a microfluidic culture chip, such as that depicted in FIG. 1A. The microfluidic culture chip 100 of FIG. 1A has at least one test site 102. Each test site 102 may include an elongate central matrix channel 104 configured to receive a hydrogel or other extracellular-matrix material. The central matrix channel 104 is bounded on opposing sides by one or more perfusable parallel media channels 110 that run substantially the length 128 of the test site 102. Fluid communication to the central matrix channel 104 and the one or more parallel media channels 110 is provided through one or more upper injection ports 106 and one or more lower injection ports 108, which may be used for loading matrix material, introducing cells or spheroids, and establishing medium flow or chemotactic gradients.

The parallel media channels 110 are hydrodynamically isolated from, yet diffusively coupled to, the central matrix channel 104 by an array of posts 112 arranged along each interface. The posts 112 define apertures 122 that allow molecular exchange while preventing bulk collapse of the matrix. The parallel media channels 110 may have a width 120 and height 118, and a distance 116 between them. The microfluidic culture chip 100 may have a width 124 of 25 mm, a length 126 of 75 mm, and a thickness 118 of 0.25 mm. In the enlarged view, representative spheroids 114 are shown disposed within the matrix region of channel 104 to illustrate typical culture geometry.

Particular embodiments utilizing a microfluidic culture chip include a method of screening a test compound for anti-metastatic properties including adding a tumor spheroid-collagen solution to a central matrix channel of a microfluidic culture chip; adding a first amount of a test compound media to an upper injection port of the microfluidic culture chip; adding a second amount of the test compound media to a lower injection port of the microfluidic culture chip wherein the second amount is less than the first amount; staining the microfluidic culture chip with a fluorescence compound, wherein the staining creates labeled tumor spheroids; and performing Z-stack confocal imaging using a laser scanning microscope to form Z-stacks and to assess migration of the labeled tumor spheroids within the collagen matrix thereby screening the test compound for anti-metastatic properties.

In particular embodiments, the migration is anti-gravitational migration. Anti-gravitational migration refers to any net movement of material opposite the direction of gravitational acceleration (i.e., upward displacement in a standard vertical frame where gravity acts downward). The term is descriptive and does not imply a specific mechanism or a violation of gravitational physics, rather, it captures the empirical directionality of transport against the gravitational vector. Operationally, when constituents would be expected to settle toward the lower boundary under gravity, observed upward motion is designated as anti-gravitational migration regardless of its cause. Such motion may arise from active motility (e.g., cellular propulsion), buoyancy (e.g., density lower than the surrounding medium), externally applied fields or forces (e.g., acoustic, magnetic, electric), interfacial or thermal gradients (e.g., convective flows) or bulk fluid movement.

In particular embodiments, the microfluidic culture chip has 2-10 sites per chip. In particular embodiments, the microfluidic culture chip includes posts with gaps in between on each side of the central matrix channel that are configured to retain the tumor spheroids and collagen solution. In particular embodiments, the microfluidic culture chip includes at least 2 sites per chip, each site containing injection ports for the central matrix channel and parallel media channels flanking the sites and one or more posts with gaps in between on each side of the central matrix channel separating the central matrix channel and parallel media channels.

Particular embodiments include adding tumor spheroids into a collagen solution to form a tumor spheroid-collagen solution. Tumor-spheroid collagen solution refers to a cell compatible, collagen-based pre-gel mixture formulated to suspend, embed, and/or overlay tumor spheroids for three-dimensional culture, invasion assays, or imaging. In some embodiments, the solution includes Type I collagen (e.g., acid-solubilized, pepsinized, or telopeptide-intact) optionally blended with other collagens (e.g., type III or IV) at a final concentration of 0.5-6 mg/mL, adjusted to physiological pH (e.g., 7.2-7.6) and ionic strength using one or more buffers (e.g., 1×PBS, HEPES) and neutralizing agents (e.g., NaOH). The solution may further include serum or defined supplements, adhesive ligands (e.g., RGD peptides), matrix modifiers (e.g., laminin, fibronectin, hyaluronan), growth factors, protease inhibitors, cross-linkers, or MMP-cleavable moieties to tune biochemical cues, porosity, and viscoelastic properties.

In certain examples, the tumor-spheroid collagen solution is prepared by combining a chilled acid collagen stock with a concentrated salt/buffer solution and neutralizing base, followed by dilution with sterile water or medium and gentle incorporation of pre-formed spheroids to avoid shear damage. The mixture can be cast as droplets, wells, channels, or microarray spots and polymerized to form a 3D matrix that supports spheroid maintenance and quantifiable outgrowth or invasion. The resulting gels may be characterized and/or specified by one or more parameters, including collagen mass fraction, fibril diameter and density, pore size, gelation time, optical clarity, and bulk or local stiffness (e.g., by rheology or indentation), with lot-to-lot normalization via internal standards.

Particular embodiments include adding 1 mg/mL tumor spheroid-collagen solution to the microfluidic culture chip. Particular embodiments include adding 2.5 mg/mL tumor spheroid-collagen solution to the microfluidic culture chip.

In particular embodiments, the tumor spheroids are derived from an RCC tumor. For example, RCC4 refers to a human renal cell carcinoma clear-cell subtype cell line that is widely employed as an in vitro model of von Hippel-Lindau (VHL)-deficient kidney cancer. “RCC4” encompasses parental VHL-null RCC4 cells as well as derivative lines and engineered variants. RCC4 cells may be propagated in standard mammalian tissue-culture media supplemented with serum and antibiotics using conventional methods. References to “RCC4” include cryopreserved stocks, actively proliferating cultures, and experimental derivatives suitable for assays of gene expression, ligan secretion, proliferation, apoptosis, migration, invasion, or drug screening. RCC4 cells exhibit adherent, epithelial-like morphology and a constitutive “pseudo-hypoxic” program due to loss of functional VHL, resulting in the stabilization of hypoxia-inducible factors (e.g., HIF-1α and/or HIF-2α) under normal oxygen conditions. Consequently, RCC4 cells characteristically upregulate HIF target genes and proteins, including angiogenic and metabolic factors such as VEGF, CA9, and GLUT1, and display phenotypes relevant to tumor growth, metabolic reprogramming, migration/invasion, and therapy response.

In particular embodiments, the tumor spheroids are A498 spheroids. A498 cancer cells are a widely used human renal cell carcinoma line derived from a primary kidney tumor of a 52-year-old female. A498 cells are commonly treated as a clear-cell RCC (ccRCC) model because they carry loss-of-function alterations in VHL (often reported as a VHL frameshift), leading to constitutive HIF pathway activity and ccRCC-like biology.

In particular embodiments, the tumor spheroids are HEPG2 spheroids. HEPG2 cancer cells are an adherent human liver cancer line widely used as a hepatocyte-like model for metabolism and toxicity studies, used as a control cell type within the current disclosure. Although HEPG2 cancer cells have been labeled as hepatocellular carcinoma (HCC) or as hepatoblastoma-like, they retain robust hepatic functions under either label. HEPG2 cells secrete albumin, transferrin, apolipoproteins, and α-fetoprotein, display functional LDL receptor activity, and support studies of lipoprotein assembly and glucose/lipid metabolism. HEPG2 cells are known to express many drug-metabolizing enzymes. HEPG2 cells are typically p53 wild-type, HBV-negative and not naturally permissive to HBV infection (lack NTCP), have a hyper-triploid karyotype, and are generally non-tumorigenic in nude mice. In culture, HEPG2 cells are known to grow as epithelial clusters in MEM/DMEM with 10% FBS, making them a convenient, stable platform for toxicology, pharmacology, metabolism, and secretion readouts.

Particular embodiments include polymerizing collagen. Polymerizing collagen refers to inducing collagen fibrillogenesis to form a continuous, three-dimensional hydrogel network from a soluble collagen precursor, such as acid-solubilized type 1 collagen, atelo- or telo-collagen. References to “polymerizing collagen” include neutralization-triggered fibrillogenesis of type I collagen and compatible variants (e.g., type III or IV blends), and encompass casting as droplets, slabs, microwells, or microfluidic channels to yield hydrogels suitable for imaging, invasion assays, or drug-response studies. Polymerization can be initiated by neutralizing an acidic stock to near-physiological pH and ionic strength and incubating at a temperature sufficient to promote fibril assembly. In some embodiments, the collagen is maintained on ice prior to use, combined with a concentrated buffer/salt solution (e.g., 10×PBS or HEPES-buffered saline) and a base (e.g., NaOH) to achieve a final pH of 7.2-7.6 and an ionic strength of 0.1-0.2 M, and then incubated at a temperature of 20-37° C. to polymerize for 10-60 minutes (e.g., 30-45 minutes at 37° C.). The final collagen concentration can be 0.5-6 mg/mL (e.g., 1-3 mg/mL) to tune gel stiffness, porosity, and fiber architecture. In certain examples, polymerization is monitored by the onset of turbidity (e.g., 300-320 nm absorbance increase), by the development of a storage modulus exceeding the loss modulus, or by visual gelation in a well or channel.

Optionally, polymerization conditions are adjusted to control microstructure and mechanics, including varying collagen concentration, pH, incubation temperature, and salt composition; adding co-factors (e.g., fibronectin, laminin), neutral-buffer systems (e.g., HEPES, TRIS), or crowding agents; or pre-seeding cells or spheroids prior to gelation. Post-polymerization stabilization may include enzymatic or chemical cross-linking (e.g., genipin, EDC/NHS), thermal annealing, or controlled dehydration/re-hydration.

In some embodiments, polymerization is performed at 37° C. for: 10-60 minutes, 20-40 minutes, or for at least 30 minutes. Particular embodiments include polymerizing the collagen solution at 37° C. for 10-60 minutes to form a collagen matrix. Particular embodiments include polymerizing the collagen at 37° C. for 20-40 minutes to form a collagen matrix. Particular embodiments include polymerizing the collagen at 37° C. for 30 minutes to form a collagen matrix.

In particular embodiments, the collagen matrix is biomimetic collagen matrix. “Biomimetic collagen” refers to materials engineered to mimic the structure and function of native collagen, without relying on animal-derived collagen. Exemplary structures and functions mimicked include a triple-helical conformation, a capacity to self-assemble into fibrils or fibril-like bundles, a cell-adhesive bioactivity, and an enzymatic degradability or protease resistance. Exemplary biomimetic collagens include recombinant human collagen produced in a heterologous host, collagen-mimetic peptides (CMPs), engineered collagen-like proteins, hybrid polymer scaffolds, and CMP-functionalized nanoparticles.

The number of spheroids in a collagen-RCC tumor spheroid matrix can vary. In some embodiments, the number of spheroids in the collagen-RCC tumor spheroid matrix is 8-16 tumor spheroids/μL. In other embodiments the collagen-RCC tumor spheroid matrix has at least 8 tumor spheroids/μL, at least 9 tumor spheroids/μL, at least 10 tumor spheroids/μL, at least 11 tumor spheroids/μL, at least 12 tumor spheroids/μL, at least 13 tumor spheroids/μL, at least 14 tumor spheroids/μL, at least 15 tumor spheroids/μL, or at least 16 tumor spheroids/μL.

Particular embodiments include adding a test compound media to an injection port. Test compound refers to any material or entity evaluated for an effect in the disclosed assays. Test compounds may include, for example, small molecules, peptides, proteins, antibodies or antigen-binding fragments, nucleic acids (e.g., siRNA, ASO, mRNA, plasmids), gene-editing reagents, carbohydrates, lipids, natural-product extracts or fractions, polymeric materials, nanoparticles or liposomes, metabolites, cells, and combinations thereof.

Test compounds may be selected in a variety of ways. In some examples, the test compounds are drawn from a library, such as diversity-oriented, focused target-class, FDA-approved/repurposing, fragment, peptide, CRISPR/ORF, or natural-product collections, which may be screened in arrayed or pooled formats. In other examples, the test compounds are chosen at random from an available set to minimize selection bias and enable unbiased discovery. Test compounds may also be selected based on particular characteristics, such as target engagement, pathway membership (e.g., EMT, Rho/ROCK, or PI3K pathways), physicochemical properties, structural motifs suggested by prior structure-activity relationships, predicted ADME/T liabilities, or prior phenotypic evidence. Additional selection routes include structure- or ligand-based virtual screening, AI/ML-based ranking from multimodal datasets, literature- or clinician-curated candidates, patient-derived factors (e.g., serum or exosomes), rational combinations intended to produce synergy with a benchmark agent, medicinal-chemistry analog series, and deconvolution of active fractions from natural extracts.

In particular embodiments, the test compound can be a drug or a putative anti-migratory compound. “Anti-migratory compound” refers to any agent that, under defined assay conditions, reduces the directed or random movement of cells without needing to kill them. “Anti-migratory” generally describes a mechanistic/phenotypic effect. For example, a compound is deemed anti-migratory in 3D test spaces if it reduces group displacement, invasion depth, front velocity, or area/volume of spread relative to controls at viability-preserving doses. Outcomes may include decreased centroid shift, lower mean squared displacement, smaller isosurface expansion, or reduced number/length of invasive strands. In other examples, a compound is deemed anti-migratory with regards to cancer metastasis if it impairs steps of the metastatic cascade that require mobility—local invasion, intravasation, survival/extravasation, and early colonization—leading to fewer or delated distant lesions in appropriate models at exposures that preserve general health. Exemplary anti-migratory compounds include FAK/integrin pathway inhibitors (e.g., defactinib, cilengitide), Src family kinase inhibitors (e.g., dasatinib), chemokine-axis blockers (e.g., plerixafor), and matrix-remodeling inhibitors (e.g., marimastat, batimastat).

In particular embodiments, a test compound within a test compound media is a drug. In particular embodiments, the drug is bortezomib. “Bortezomib” encompasses the free base or pharmaceutically acceptable salts, stereoisomers, solvates, prodrugs, and formulations suitable for the intended route of administration. Bortezomib is a reversible, dipeptidyl boronic-acid inhibitor of the 26S proteasome, which preferentially blocks the chymotrypsin-like activity of the 20S core particle. By impairing proteasomal degradation of ubiquitinated proteins, bortezomib induces proteotoxic stress, stabilizes pro-apoptotic factors, and attenuates NF-κB signaling through prevention of IκB degradation. Plasma cells are particularly sensitive to this mechanism due to high immunoglobulin synthesis burden. Clinically, bortezomib is an antineoplastic approved for the treatment of multiple myeloma and mantle cell lymphoma, and is commonly used in combination regimens (e.g., with dexamethasone and/or immunomodulatory agents). In experimental contexts, bortezomib can serve as a positive control or mechanistic probe for proteasome inhibition, apoptosis induction, of NF-κB pathway suppression. It may be applied to systems described herein to evaluate effects on viability, proliferation, stress responses, or cell migration/invasion phenotypes, typically with appropriate vehicle controls and concentration-response testing.

In particular embodiments, the test compound includes Saracatinib, Defactinib, latrunculin A, Blebbistatin, RKI-1447, AT13148, or ISO-1.

Saracatinib is an ATP-competitive small-molecule inhibitor of the Src family of kinases (SFKs), including Src, Fyn, Lyn, and Yes, with activity that attenuates downstream signaling pathways involved in cytoskeletal dynamics, focal adhesion turnover, invadopodia formation, and cell motility. “Saracatinib” refers to the free base or a pharmaceutically acceptable salt and includes stereochemical forms, solvates, prodrugs, and formulations suitable for the intended route of administration. By suppressing SFK activity (and associated crosstalk with focal adhesion kinase, PI3K/AKT, and MAPK), Saracatinib can reduce migration and invasion, impair tumor-stroma interactions (e.g., osteoclast-mediated bone resorption), and modulate survival signaling. In some examples, Saracatinib may serve as a mechanistic probe or positive control for SFK inhibition and anti-migratory/anti-invasive effects. It can be applied to systems described herein to assess effects on metrics such as collective migration speed, directional persistence, invasion depth, outgrowth radius, focal adhesion dynamics and viability.

Defactinib is an ATP-competitive small-molecule inhibitor that targets focal adhesion kinase (FAK/PTK2) and, at relevant concentrations, may inhibit the related kinase PYK2 (PTK2B). “Defactinib” encompasses the free base or pharmaceutically acceptable salts, stereoisomers, solvates, prodrugs, and formulations suitable for the intended route of administration. By suppressing FAK catalytic activity and downstream signaling (e.g., FAK-Src-paxillin axes; crosstalk with PI3K/AKT, MAPK, and Rho GTPases), defactinib modulates focal-adhesion turnover, actin cytoskeletal dynamics, mechano-transduction, and transcriptional programs linked to YAP/TAZ. Functionally, this can reduce tumor-cell migration and invasion, alter epithelial-mesenchymal plasticity, and impact the tumor microenvironment (e.g., cancer-associated fibroblast contractility, immune infiltration). In experimental contexts, defactinib can serve as a mechanistic probe or positive control for FAK inhibition and anti-migratory/anti-invasive effects. It may be applied to systems described herein to assess endpoints such as collective migration speed, directional persistence, invasion depth, outgrowth radius, focal-adhesion dynamics, and viability. Orthogonal readouts can include reduced phosphorylation of FAK at Y397 (autophosphorylation) and downstream effectors, as well as shifts in YAP/TAZ localization or target-gene expression.

Latrunculin A (“LatA”) is a marine macrolide toxin that binds monomeric (G-)acting in a 1:1 complex, sequestering actin monomers and thereby inhibiting actin polymerization. “Latrunculin A” encompasses the compound as obtained from commercial or purified sources and, where applicable, pharmaceutically acceptable salts or formulations suitable for the intended route of administrations. Functionally, LatA destabilizes filamentous (F-)actin networks, leading to rapid disruption of stress fibers, focal-adhesion turnover, altered membrane tension, impaired endocytosis/exocytosis, and attenuation of protrusive structures (e.g., lamellipodia, filopodia, invadopodia). In migratory and invasive cell models, LatA produces characteristic anti-migratory phenotypes, such as reduced cell spreading, lower migration speed/persistence, and diminished invasion, often with reversible effects upon washout as actin monomer pools rebalance. In experimental contexts, LatA is widely used as a tool compound for acute actin perturbation and as a positive control for actin-dependent processes. It may be applied to systems described herein to assess endpoints such as collective migration speed, invasion depth, outgrowth radius, focal-adhesion dynamics, and viability. LatA preparations are handled as light-sensitive DMSO stocks and diluted freshly into assay media, wherein cytotoxicity increases with dose and duration. For mechanistic specificity controls, compounds with distinct actin mechanisms may be included—e.g., cytochalasin D (barbed-end capping) or jasplakinolide (F-actin stabilization)—to differentiate monomer-sequestration effects from filament capping or stabilization.

Blebbistatin is a small-molecule inhibitor of class II myosins that selectively suppresses the ATPase activity of non-muscle myosin II (e.g., IIA, IIB, IIC) and many muscle isoforms by stabilizing a myosin conformation with reduced acting affinity and impaired phosphate release. “Blebbistatin” encompasses the racemate, active enantiomer, pharmaceutically acceptable salts, solvates, and formulations suitable for the intended route of administration. Functionally, blebbistatin diminishes actomyosin contractility, leading to loss of stress fibers, reduced focal-adhesion maturation and traction forces, altered cortical tension, and characteristic defects in cytokinesis. In motility assays, this manifests as anti-contractile, often anti-migratory or anti-invasive phenotypes in cell types and matrices where myosin II-dependent force generation is rate-limiting; in some contexts it can also uncouple protrusion from contraction, increasing lamellipodial activity but decreasing net translocation. In experimental use, blebbistatin serves as a mechanistic probe or positive control for myosin II inhibition across systems described herein, with readouts that may include collective migration speed, directional persistence, invasion depth, outgrowth radius, focal-adhesion dynamics, traction force, and viability.

RKI-1447 refers to a small-molecule, ATP-competitive inhibitor or Rho-associated coiled-coil-containing kinases 1 and 2 (ROCK1/ROCK2). “RKI-1447” encompasses the compound as obtained from commercial or synthesized sources and, where applicable, pharmaceutically acceptable salts, stereochemical forms, solvates, prodrugs, and formulations suitable for the intended route of administration. By suppressing ROCK catalytic activity, RKI-1447 diminishes phosphorylation of canonical substrates such as myosin light chain (MLC) and MYPT1, thereby lowering actomyosin contractility, stress-fiber formation, and focal-adhesion maturation. Functionally, ROCK inhibition can reduce cell and tissue tension, alter mechano-transduction (e.g., YAP/TAZ programs), and produce characteristic anti-migratory and anti-invasive phenotypes in models where RhoA-ROCK signaling drives motility, invasion, or matrix remodeling. In experimental contexts, RKI-1447 may serve as a mechanistic probe or positive control for ROCK pathway inhibition. It can be applied to systems described herein to assess endpoints such as collective migration speed, directional persistence, invasion depth, outgrowth radius, focal-adhesion dynamics, traction forces, cytoskeletal organization, and viability. Orthogonal confirmation of target engagement typically includes reduced phospho-MLC and phospho-MYPT1, stress-fiber loss (phalloidin staining), and decreased cell traction.

AT13148 refers to an ATP-competitive multi-AGC kinase inhibitor with activity against ROCK1/ROCK2 and class-typical AGC family members (e.g., AKT/PKB isoforms, p70S6K, PKA/PKC to varying degrees, assay-dependent). “AT13148” encompasses the free base or pharmaceutically acceptable salts, stereochemical forms, solvates, prodrugs, and formulations suitable for the intended route of administration. Through inhibition of ROCK, AT13148 lowers phosphorylation of myosin regulatory light chain (MLC) and MYPT1, thereby reducing actomyosin contractility, stress-fiber assembly, focal-adhesion maturation, and tissue-level tension. Concomitant inhibition of AKT signaling can attenuate survival and growth pathways (e.g., reduced phosphorylation of AKT substrates such as GSK3β and PRAS40, with downstream effects on mTOR/S6K axes). Functionally, these combined activities can produce anti-migratory and anti-invasive phenotypes, alter mechano-transduction (including YAP/TAZ programs), and—at sufficient exposure—impact proliferation and viability. In experimental contexts, AT13148 serves as a mechanistic probe or positive control where coordinated inhibition of ROCK and AKT-linked signaling is desired. It may be applied to systems described herein to assess endpoints such as collective migration speed, directional persistence, invasion depth, spheroid outgrowth radius, focal-adhesion dynamics, traction forces, cytoskeletal organization, and cell viability. Orthogonal confirmation of target engagement can include decreases in p-MLC and p-MYPT1 (ROCK pathway) together with reductions in p-AKT (S473/T308), p-GSK3β, and p-S6 (AKT/mTOR axis).

ISO-1 refers to a small-molecule macrophage migration inhibitory factor (MIF) antagonist that binds the tautomerase/ligand-binding pocket of MIF and disrupts MIF-dependent signaling. “ISO-1” encompasses commercially obtained or synthesized material and, where applicable, pharmaceutically acceptable salts, stereochemical forms, solvates, prodrugs, and formulations suitable for the intended route of administration in the relevant model system. By attenuating MIF engagement of receptors such as CD74 (often with CD44 co-receptor) and chemokine receptors (e.g., CXCR2/CXCR4 contexts), ISO-1 can blunt downstream pathways including ERK/MAPK, PI3K/AKT, and NF-κB, reduce pro-inflammatory cytokine production, and modulate cell survival, motility, and immune-tumor interactions. In experimental use, ISO-1 may serve as a mechanistic probe or positive control for MIF pathway inhibition. It can be applied to systems described herein to assess endpoints such as collective migration speed, directional persistence, invasion depth, spheroid outgrowth, matrix remodeling, cytokine/chemokine secretion, and viability.

Genetic engineering of cells can be achieved using any appropriate technique. For example, desired genes or genetic constructs disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence that encodes a molecule of interest (e.g., a CAR, eTCR, interfering RNA, mRNA, etc). This definition includes various sequence polymorphisms, mutations, and/or sequence variants wherein such alterations do not substantially affect the function of the encoded genetic construct. The term “gene” may include not only coding sequences but also regulatory regions such as promoters, enhancers, and termination regions. Gene sequences encoding the molecule can be DNA or RNA that directs the expression of the genetic construct. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

Gene sequences encoding a molecule of interest can be readily prepared by synthetic or recombinant methods from relevant amino acid or RNA. In particular embodiments, the gene sequence encoding a molecule of interest can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

“Encoding” refers to the property of specific sequences of nucleotides in a gene, such as a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed genetic constructs can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et ah, Mol. Ther. 17(8): 1453-1464 (2009). Other examples of lentivirus vectors that may be used in the clinic, include: the LENTIVECTOR® gene delivery technology from Oxford BioMedica, the LENTIMAX™ vector system from Lentigen and the like. Nonclinical types of lentiviral vectors are also available and would be known to one skilled in the art. In particular embodiments, cells are genetically engineered to express a molecule of interest using a lentivirus or lentiviral vector.

Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. A zinc finger is a domain of 30 amino acids within the zinc finger binding domain whose structure is stabilized through coordination of a zinc ion. Examples of zinc fingers include C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. A well-known example of a ZFN is a fusion of the FokI nuclease with a zinc finger DNA binding domain. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(I):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of a genetic construct into cells. Generally, such methods will involve introducing into cells (i) a first vector encoding a transposase (or a transposase polypeptide) and (ii) a second vector encoding a desired genetic element that is flanked by transposon repeats. Transposons or transposable elements include a (short) nucleic acid sequence with terminal repeat sequences upstream and downstream thereof and encode enzymes that facilitate the excision and insertion of the nucleic acid into target DNA sequences.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7,148,203; 8,227,432; and 9,228,180.

In an exemplary embodiment, the test compound is applied to the assay system (e.g., 3D migration, invasion) to determine whether it modulates one or more predefined metrics, such as collective migration speed, directional persistence, invasive-front positions, radial outgrowth, while simultaneously monitoring viability. For drug candidates or putative anti-migratory compounds, activity may be indicated by a reduction in collective migration or invasion at concentrations that do not cause unacceptable cytotoxicity; for immune-cell test articles, activity may include altered tumor-cell motility, direct cytolysis, or extracellular-matrix remodeling.

Particular embodiments include adding 50-100 μL of the test compound media in the upper injection port. Particular embodiments include adding 25-75 μL of the test compound media in the lower injection port. Particular embodiments include adding 70 μL of the test compound media in the upper injection port. Particular embodiments include adding 50 μL of the test compound media in the lower injection port. Particular embodiments include a concentration of the test compound of at least 1 ng/μL up to 1 g/μL.

Particular embodiments include staining cells within the tumor spheroids. In particular embodiments, the staining occurs 20-28 hours after the adding to the lower injection port.

Particular embodiments include creating a Montage gallery of z-stack positions of labeled tumor spheroids. “Montage Gallery” refers to a composite, tiled arrangement of multiple related images displayed within a single field for rapid visual comparison and documentation. References to a “montage gallery” include automatically generated contact sheets used for quality control (e.g., focus, photobleaching, drift), selection of representative fields for publication, and archival records of acquisition parameters, as well as curated, annotated composites prepared for analysis or reporting. In general, a montage gallery may present frame from a time series, different channels (e.g., fluorescence colors), fields of view, or parameter conditions (e.g., dose, time, treatment), organized in a pre-defined grid or an auto-fit contact sheet. Each tile can optionally include labels, scale bars, color legends, regions-of-interest (ROI) outlines, and/or metadata (e.g., acquisition time, channel, objective, exposure), and may be rendered at uniform pixel density to preserve relative size. In some embodiments montages are exported as high-resolution bitmaps (e.g., TIFF, PNG) or vector-embedded formats (e.g., PDF) with selectable compression, bit depth, and ICC color profiles to maintain quantitative fidelity. In the context of laser-scanning microscopy, a “montage gallery” of compiled z-stacks denotes a tiled representation of depth-resolved image planes from one or more stacks, optionally including (i) sequential axial slices (z-planes) shown in order; (ii) derived projections (e.g., maximum-intensity, average, or sum projections); (iii) orthogonal views (x-z, y-z reslices); and/or (iv) stitched mosaics spanning adjacent tiles/tile-scan positions

“Z-stack” refers to a set of sequential optical sections of the same field of view acquired at incrementally spaced focal depths along the optical (z) axis of a microscope. References to a “z-stack” encompass raw axial planes, processed derivatives (e.g., deconvolved stacks; drift-corrected and registered stacks), and depth-reduced renderings such as maximum-intensity, average, or sum projections, as well as orthogonal reslices (x-z, y-z) and 3D reconstructions/segmentations generated therefrom. Each plane in the stack represents image data from a defined axial position separated by a known step size, yielding a volumetric dataset with voxels characterized by lateral sampling and axial sampling. Z-stacks may be captured on later-scanning confocal, spinning-disk, widefield, multiphoton, or light-sheet instruments, and can include simple or multiple imaging channels and, in some embodiments, repeated over time. Acquisition parameters can include objective NA, refractive index, pinhole size, detector settings, and sampling chosen to satisfy Nyquist criteria for the system point-spread function.

In some embodiments, z-stacks are organized into montage galleries for quality control or comparative analysis, with consistent scale bars, axial spacing annotations, and metadata preserved to maintain quantitative interpretability. Potential artifacts (e.g., spherical aberration from refractive-index mismatch, photobleaching with depth, or axial scaling errors) may be corrected or documented during processing to ensure reproducibility across samples and experiments.

The size of the Z-stacks can vary depending on experimental design. “Z-stack size”, “Z-stack span”, or other equivalents refer to the total axial span of the Z-axis. In some embodiments, the z-stack size spans at least 100 μm, at least 110 μm, or at least 120 μm. The Z-stack size can further be divided into a Z-stack step size. In some embodiments, the Z-stacks span 100-120 μm. In other embodiments, the Z-stacks span at least 100 μm, at least 110 μm, or at least 120 μm. In particular embodiments, the z-stacks span at least 100 μm. In particular embodiments, the z-stacks span at least 110 μm. In particular embodiments, the z-stacks span at least 120 μm.

“Step size” refers to the axial distance, along the microscope optical axis, between the acquisition positions of successive optical sections in a Z-stack (i.e., the number of images captured over the Z-stack size). Step size is expressed in units of length (e.g., micrometers) and may refer to the commanded interval set in the acquisition software and/or the actual interval realized at the specimen, which can be verified from image metadata or calibration standards. Step size can be uniform across the stack used to generate a montage and is determined from the start and end Z positions divided by the number of intervals between planes. Step size is selected with regard to the system's axial point-spread function and the intended analysis. Step size may be fixed or adaptively varied (e.g., finer spacing near features of interest). Step size may be corrected for refractive-index mismatch between immersion medium and specimen. The step size distance may vary based on experimental design and/or mechanistic properties.

In particular embodiments, the z-stacks span a 2-10 μm step size. In particular embodiments, the z-stacks span a 4-8 μm step size. In particular embodiments, the z-stacks span a 5-6 μm step size. In particular embodiments, the z-stacks span a 5.5 μm step size.

The time of creating a Montage gallery can vary. In some embodiments, the time of creating is at least 1 hour after adding a test solution, at least 2 hours after adding a test solution, at least 3 hours after adding a test solution, at least 4 hours after adding a test solution, at least 5 hours after adding a test solution, at least 6 hours after adding a test solution, at least 7 hours after adding a test solution, at least 8 hours after adding a test solution, at least 9 hours after adding a test solution, or at least 10 hours after adding a test solution. In some embodiments, the time of creating is within 10 hours of adding a test solution. In other embodiments, the time of creating is within 5 minutes of adding a test solution. In yet other embodiments, the time of creating is every hour for 24 hours after adding a test solution or every hour for 48 hours after adding a test solution.

In particular embodiments, the creating is within 1 hour after adding the tumor spheroid-collagen solution to the central matrix channel of the microfluidic culture chip. In particular embodiments, the creating is within 10 hours after adding the tumor spheroid-collagen solution to the central matrix channel of the microfluidic culture chip. In particular embodiments, the creating is after 1 hour, 2 hours, 3 hours, 4 hours, and 5 hours after adding the tumor spheroid-collagen solution to the central matrix channel of the microfluidic culture chip and within 10 hours after adding the tumor spheroid-collagen solution to the central matrix channel of the microfluidic culture chip. In particular embodiments, the creating is within 5 minutes after adding the tumor spheroid-collagen solution to the central matrix channel of the microfluidic culture chip. In particular embodiments, the creating is every hour for 24 or 48 hours after adding the tumor spheroid-collagen solution to the central matrix channel of the microfluidic culture chip.

In particular embodiments, the Z-stacks are obtained from a bottom of the collagen matrix. In particular embodiments, the bottom of the collagen matrix is marked by fluorescence. In particular embodiments, the bottom of the collagen matrix is marked by fluorescence of one or more posts separating the central matrix channel and parallel media channels.

“Fluorescence” refers to the light emission from a molecule, complex, nanoparticle, or other emissive species (“fluorophore”) that occurs following the absorption of excitation radiation and subsequent radiative relaxation from an excited electronic state to a lower-energy state. Fluorescent emission typically exhibits a wavelength longer than the excitation wavelength (Stokes shift), with intensity and spectral characteristics determined by the fluorophore's environment, concentration, excitation power, and intrinsic properties such as quantum yield and fluorescence lifetime. “Fluorescence” encompasses steady-state and time-resolved emission, single- and multi-photon excitation, and emissive readouts acquired by microscopy, flow cytometry, spectroscopy, or plate-based detection. In some embodiments, fluorescence is generated from endogenous (“autofluorescence”) or exogenous labels including organic dyes, fluorescent proteins, rare-earth or quantum-dot emitters, and environment-sensitive probes. Detection may employ appropriate excitation sources (e.g., lasers, LEDs), optical filters or dispersive elements to isolate excitation and emission bands, and detectors (e.g., PMTs, sCMOS, APDs). References to “fluorescence” include raw intensity data, spectrally unmixed or lifetime-resolved representations, and derivative metrics (e.g., ratio images, FRET efficiencies) suitable for qualitative visualization or quantitative analysis.

In particular embodiments, a script-based automated system quantifies at least one of a cell invasion, a cell migration, or a cell death. “Script-based automated system” refers to a reproducible, code-driven workflow that orchestrates all steps required to convert raw Z-stack confocal image data into analysis outputs and presentation montages—without manual intervention beyond launch. The custom scripts (e.g., Fiji/ImageJ macros, Python, R, or MATLAB) automatically open image files, parse metadata, split channels, register data, apply standardized preprocessing (e.g., background subtraction, deconvolution/denoising), segment 3D objects, and perform region of interest (ROI) analysis under fixed and/or versioned parameters. The scripts then execute quantification routines, including volume summation of segmented 3D objects, and write both per-object and per-image metrics to a summary table alongside run longs. As part of the script-based automated system, the system composes montages for Z-stacks (e.g., maximum-intensity projections, orthogonal views, slice grids, and channel-merged panels), scales them uniformly, annotates scale bars, and names files deterministically using a template that encodes sample ID, channel(s), step size, Z-range, processing parameters, and time stamp.

In particular embodiments, the script-based automated system is unbiased. “Unbiased” refers to a script-based system that executes identical, pre-specified operations on every image/stack without outcome-dependent human choices (i.e., no cherry-picking, hand-turning per sample, or parameter tweaks after seeing results). “Unbiased” speaks to process impartiality and reproducibility, not to zero measurement error; accuracy still depends on calibration and validation. All steps—loading, preprocessing, channel handling, segmentation, ROI logic, quantification, montage assembly, and file naming—are parameterized and locked (e.g., via a config file or versioned core), run deterministically, and applied uniformly across conditions, batches, and replicates.

The current disclosure also includes systems. Particular embodiments include a system for screening a compound for anti-metastatic properties against renal cell carcinoma (RCC), the system including: a microfluidic culture chip, wherein the microfluidic culture chip includes at least 2 sites per chip, each site containing injection ports for a central matrix channel and parallel media channels flanking the sites, and posts with gaps in between on each side of the matrix channel separating the central matrix channel and parallel media channels; a collagen-RCC tumor spheroid matrix in the central matrix channel of the microfluidic culture chip, wherein collagen within the collagen-RCC tumor spheroid matrix is polymerized and includes 4-20 tumor spheroids/μL; and an upper injection port and a lower injection port, each including a test compound media with a pressure gradient between the upper injection port and the lower injection port.

In particular embodiments, the microfluidic culture chip has 2-10 sites per chip. In particular embodiments, the microfluidic culture chip includes posts with gaps in between on each side of the central matrix channel that are configured to retain the tumor spheroids and collagen solution. In particular embodiments, the microfluidic culture chip includes at least 2 sites per chip, each site containing injection ports for the central matrix channel and parallel media channels flanking the sites and one or more posts with gaps in between on each side of the central matrix channel separating the central matrix channel and parallel media channels.

In particular embodiments, tumor spheroids within the collagen-RCC tumor spheroid matrix are A498 spheroids. In particular embodiments, tumor spheroids within the collagen-RCC tumor spheroid matrix are HEPG2 spheroids.

In particular embodiments, tumor spheroids within the collagen-RCC tumor spheroid matrix have 100-500 cells/spheroid. In particular embodiments, tumor spheroids within the collagen-RCC tumor spheroid matrix have 125 cells/spheroid. In particular embodiments, tumor spheroids within the collagen-RCC tumor spheroid matrix have 120 cells/spheroid.

In particular embodiments, the collagen-RCC tumor spheroid matrix has 8-16 tumor spheroids/μL. In particular embodiments, the collagen-RCC tumor spheroid matrix has 12 tumor spheroids/μL.

In particular embodiments, collagen within the collagen-RCC tumor spheroid matrix was polymerized at 37° C. for 10-60 minutes. In particular embodiments, collagen within the collagen-RCC tumor spheroid matrix was polymerized at 37° C. for 20-40 minutes. In particular embodiments, collagen within the collagen-RCC tumor spheroid matrix was polymerized at 37° C. for 30 minutes. In particular embodiments, collagen within the collagen-RCC tumor spheroid matrix is a biomimetic collagen.

In particular embodiments, the system includes 50-100 μL of the test compound media in the upper injection port. In particular embodiments, the system includes 25-75 μL of the test compound media in the lower injection port. In particular embodiments, the system includes 70 μL of the test compound media in the upper injection port. In particular embodiments, the system includes 50 μL of the test compound media in the lower injection port.

In particular embodiments, the test compound media includes a drug. In particular embodiments, the drug is bortezomib. In particular embodiments, the test compound media includes a putative anti-migratory compound. In particular embodiments, a test compound within the test compound media includes Saracatinib, Defactinib, latrunculin A, Blebbistatin, RKI-1447, AT13148, or ISO-1.

In particular embodiments of the systems, the bottom of the collagen-RCC tumor spheroid matrix is marked by fluorescence. In particular embodiments, the bottom of the collagen-RCC tumor spheroid matrix is marked by fluorescence of one or more posts separating the central matrix channel and parallel media channels.

Particular embodiments of the systems include a processor that executes a script-based automated quantification at least one of a cell invasion, a cell migration, or a cell death. In particular embodiments, the script-based automated quantification is unbiased.

### EXEMPLARY EMBODIMENTS

1. A method of screening a test compound for anti-metastatic properties, the method including:
   adding a tumor spheroid-collagen solution to a central matrix channel of a microfluidic culture chip;
   polymerizing collagen within the tumor spheroid-collagen solution to form a collagen matrix;
   adding a first amount of a test compound media to an upper injection port of the microfluidic culture chip;
   adding a second amount of the test compound media to a lower injection port of the microfluidic culture chip wherein the second amount is less than the first amount;
   staining the microfluidic culture chip with a fluorescence compound, wherein the staining creates labeled tumor spheroids; and
   performing Z-stack confocal imaging using a laser scanning microscope to form Z-stacks and to assess migration of the labeled tumor spheroids within the collagen matrix thereby screening the test compound for anti-metastatic properties.
2. The method of embodiment 1, wherein the migration is anti-gravitational migration.
3. The method of embodiment 1 or 2, where in the microfluidic culture chip has 2-10 sites per chip.
4. The method of any of embodiments 1-3, where in the microfluidic culture chip includes posts with gaps in between on each side of the central matrix channel that are configured to retain the tumor spheroids and collagen solution.
5. The method of any of embodiments 1-4, wherein the microfluidic culture chip includes at least 2 sites per chip, each site containing injection ports for the central matrix channel and parallel media channels flanking the sites and one or more posts with gaps in between on each side of the central matrix channel separating the central matrix channel and parallel media channels.
6. The method of any of embodiments 1-5, further including adding tumor spheroids into a collagen solution to form the tumor spheroid-collagen solution.
7. The method of any of embodiments 1-6, wherein the tumor spheroids are derived from an RCC tumor.
8. The method of any of embodiments 1-7, wherein the tumor spheroids are A498 spheroids.
9. The method of any of embodiments 1-6, wherein the tumor spheroids are HEPG2 spheroids.
10. The method of any of embodiments 1-9, wherein the tumor spheroids have 100-500 cells/spheroid.
11. The method of any of embodiments 1-9, wherein the tumor spheroids have 125 cells/spheroid.
12. The method of any of embodiments 1-9, wherein the tumor spheroids have 120 cells/spheroid.
13. The method of any of embodiments 1-12, including adding 1 mg/mL tumor spheroid-collagen solution to the microfluidic culture chip.
14. The method of any of embodiments 1-12, including adding 2.5 mg/mL tumor spheroid-collagen solution to the microfluidic culture chip.
15. The method of any of embodiments 1-14, wherein polymerizing collagen within the tumor spheroid-collagen solution is for 10-60 minutes at 37° C.
16. The method of any of embodiments 1-14, wherein polymerizing collagen within the tumor spheroid-collagen solution is for 20-40 minutes at 37° C.
17. The method of any of embodiments 1-14, wherein polymerizing collagen within the tumor spheroid-collagen solution is for 30 minutes at 37° C.
18. The method of any of embodiments 1-17, wherein the collagen matrix is a biomimetic collagen matrix.
19. The method of any of embodiments 1-18, including adding 50-100 μL of the test compound media in the upper injection port.
20. The method of any of embodiments 1-18, including adding 25-75 μL of the test compound media in the lower injection port.
21. The method of any of embodiments 1-18, including adding 70 μL of the test compound media in the upper injection port.
22. The method of any of embodiments 1-18, including adding 50 μL of the test compound media in the lower injection port.
23. The method of any of embodiments 1-22, wherein the staining occurs 20-28 hours after the adding to the lower injection port.
24. The method of any of embodiments 1-23, wherein a test compound within the test compound media is a drug.
25. The method of embodiment 24, wherein the drug is bortezomib.
26. The method of any of embodiments 1-23, wherein the test compound is a putative anti-migratory compound.
27. The method of any of embodiments 1-23, wherein the test compound includes Saracatinib, Defactinib, latrunculin A, Blebbistatin, RKI-1447, AT13148, or ISO-1.
28. The method of any of embodiments 1-27, further including creating a Montage gallery of each z-stack position of labeled tumor spheroids.
29. The method of embodiment 28, wherein the creating is within 1 hour after adding the tumor spheroid-collagen solution to the central matrix channel of the microfluidic culture chip.
30. The method of embodiment 28, wherein the creating is within 10 hours after adding the tumor spheroid-collagen solution to the central matrix channel of the microfluidic culture chip.
31. The method of embodiment 28, wherein the creating is after 1 hour, 2 hours, 3 hours, 4 hours, and 5 hours after adding the tumor spheroid-collagen solution to the central matrix channel of the microfluidic culture chip and within 10 hours after adding the tumor spheroid-collagen solution to the central matrix channel of the microfluidic culture chip.
32. The method of embodiment 28, wherein the creating is within 5 minutes after adding the tumor spheroid-collagen solution to the central matrix channel of the microfluidic culture chip.
33. The method of embodiment 28, wherein the creating is every hour for 24 or 48 hours after adding the tumor spheroid-collagen solution to the central matrix channel of the microfluidic culture chip.
34. The method of any of embodiments 1-33, wherein the Z-stacks are obtained from a bottom of the collagen matrix.
35. The method of embodiment 34, wherein the bottom of the collagen matrix is marked by fluorescence.
36. The method of embodiment 34, wherein the bottom of the collagen matrix is marked by fluorescence of one or more posts separating the central matrix channel and parallel media channels.
37. The method of any of embodiments 1-36, wherein the z-stacks span at least 100 μm.
38. The method of any of embodiments 1-36, wherein the z-stacks span at least 110 μm.
39. The method of any of embodiments 1-36, wherein the z-stacks span at least 120 μm.
40. The method of any of embodiments 1-36, wherein the z-stacks span a 2-10 μm step size.
41. The method of any of embodiments 1-36, wherein the z-stacks span a 4-8 μm step size.
42. The method of any of embodiments 1-36, wherein the z-stacks span a 5-6 μm step size.
43. The method of any of embodiments 1-36, wherein the z-stacks span a 5.5 μm step size.
44. The method of any of embodiments 1-43, wherein a script-based automated system quantifies at least one of a cell invasion, a cell migration, or a cell death.
45. The method of embodiment 44, wherein the script-based automated system is unbiased.
46. A system for screening a compound for anti-metastatic properties against renal cell carcinoma (RCC), the system including:
   a microfluidic culture chip, wherein the microfluidic culture chip includes at least 2 sites per chip, each site containing injection ports for a central matrix channel and parallel media channels flanking the sites, and posts with gaps in between on each side of the matrix channel separating the central matrix channel and parallel media channels;
   a collagen-RCC tumor spheroid matrix in the central matrix channel of the microfluidic culture chip, wherein collagen within the collagen-RCC tumor spheroid matrix is polymerized and includes 4-20 tumor spheroids/μL; and
   an upper injection port and a lower injection port, each including a test compound media with a pressure gradient between the upper injection port and the lower injection port.
47. The system of embodiment 46, where in the microfluidic culture chip has 2-10 sites per chip.
48. The system of embodiment 46 or 47, where in the microfluidic culture chip includes posts with gaps in between on each side of the central matrix channel that are configured to retain the tumor spheroids and collagen solution.
49. The system of any of embodiments 46-48, wherein the microfluidic culture chip includes at least 2 sites per chip, each site containing injection ports for the central matrix channel and parallel media channels flanking the sites and one or more posts with gaps in between on each side of the central matrix channel separating the central matrix channel and parallel media channels.
50. The system of any of embodiments 46-49, wherein tumor spheroids within the collagen-RCC tumor spheroid matrix are A498 spheroids.
51. The system of any of embodiments 46-50, wherein tumor spheroids within the collagen-RCC tumor spheroid matrix have 100-500 cells/spheroid.
52. The system of any of embodiments 46-50, wherein tumor spheroids within the collagen-RCC tumor spheroid matrix have 125 cells/spheroid.
53. The system of any of embodiments 46-50, wherein tumor spheroids within the collagen-RCC tumor spheroid matrix have 120 cells/spheroid.
54. They system of any of embodiments 46-53, wherein the collagen-RCC tumor spheroid matrix has 8-16 tumor spheroids/μL.
55. They system of any of embodiments 46-53, wherein the collagen-RCC tumor spheroid matrix has 12 tumor spheroids/μL.
56. The system of any of embodiments 46-53, wherein collagen within the collagen-RCC tumor spheroid matrix was polymerized at 37° C. for 10-60 minutes.
57. The system of any of embodiments 46-56, wherein collagen within the collagen-RCC tumor spheroid matrix was polymerized at 37° C. for 20-40 minutes.
58. The system of any of embodiments 46-56, wherein collagen within the collagen-RCC tumor spheroid matrix was polymerized at 37° C. for 30 minutes.
59. The system of any of embodiments 46-56, wherein collagen within the collagen-RCC tumor spheroid matrix is a biomimetic collagen.
60. The system of any of embodiments 46-59, including 50-100 μL of the test compound media in the upper injection port.
61. The system of any of embodiments 46-59, including 25-75 μL of the test compound media in the lower injection port.
62. The system of any of embodiments 46-59, including 70 μL of the test compound media in the upper injection port.
63. The system of any of embodiments 46-59, including 50 μL of the test compound media in the lower injection port.
64. The system of any of embodiments 46-63, wherein a test compound within the test compound media is a drug.
65. The system of embodiment 64, wherein the drug is bortezomib.
66. The system of any of embodiments 46-63, wherein a test compound within the test compound media is a putative anti-migratory compound.
67. The system of any of embodiments 46-63, wherein a test compound within the test compound media includes Saracatinib, Defactinib, latrunculin A, Blebbistatin, RKI-1447, AT13148, or ISO-1.
68. The system of any of embodiments 46-67, wherein the bottom of the collagen-RCC tumor spheroid matrix is marked by fluorescence.
69. The system of any of embodiments 46-68, wherein the bottom of the collagen-RCC tumor spheroid matrix is marked by fluorescence of one or more posts separating the central matrix channel and parallel media channels.
70. The system of any of embodiments 46-69, including a processor that executes a script-based automated quantification at least one of a cell invasion, a cell migration, or a cell death.
71. The system of embodiment 70, wherein the script-based automated quantification is unbiased.

Experimental Example. Methods Cells and Reagents. This research was approved by the Institutional Review Boards of the Fred Hutchinson Cancer Center (FHCC) and the University of Washington, and all patients provided written informed consent for the donation of deidentified samples for research. A498 and HEPG2/C3A cells were from the American Type Culture Collection (Manassas, VA). RCC4 cells were verified by the DNA Fingerprinting Service of the FHCC. Primary RCC cells were isolated from nephrectomy samples of clear cell RCC (ccRCC) using the gentleMACS Tumor Dissociation Kit (Miltenyi Biotec, Bergisch Gladbach, Germany). RCC cells were cultured in RPMI supplemented with sodium pyruvate, Lglutamine, antibiotic-antimycotic (Thermo Fisher Scientific, Waltham, MA) and 10% fetal bovine serum (Cytiva, Logan, UT). Bortezomib, defactinib, blebbistatin, AT13148, ISO-1, and RKI-1447 were from Selleck Chemicals (Houston, TX). Latrunculin A was from MilliporeSigma.

2D viability. A498 and RCC4 cells were seeded at 2.1-2.6×10⁴ cells in 1 mL in a 24-well plate in RCC media with the indicated concentration of bortezomib with a fresh media change at 24 hours. After 48 hours, 80 μL of CellTiter 96 Aqueous One Solution Cell Proliferation Assay reagent (Promega, Madison, WI) was added for 2 hours. Media was transferred to a 96-well plate and measured for absorbance at 490 nM (Synergy, Agilent).

3D chip assay. RCC cells were labeled with 24 μM CellTracker (Ct) Green CMFDA (Thermo Fisher Scientific) in serum-free media for 30 minutes, subjected to the Dead Cell Removal Kit (Miltenyi Biotech), and formed into spheroids overnight using Aggrewell 400 plates (Stem Cell Technologies) at the indicated density of cells per spheroid. Spheroids were harvested, collected at 414×g for 3 minutes, and resuspended in RCC media. A rat tail collagen I solution (Corning Life Sciences, Tewksbury, MA) was diluted in media, neutralized to pH 7.6 with 1 N NaOH, and seeded with spheroids for a final concentration of 1 or 2.5 mg/mL and 12 spheroids/μL. The collagen-spheroid solution was injected as 10 μL into the chip matrix region (Aim Biotech idenTx 3, MilliporeSigma). Collagen was polymerized at 37° C. for 30 minutes and the chips were hydrated with 70 μL in each upper port and 50 μL in each lower port with media with the indicated study drug and incubated for 48 hours with a fresh media change at 24 hours.

Confocal microscopy and image analysis. Chips were stained with media containing 3 μM DRAQ7 (BioLegend) for 2 hours followed by confocal fluorescence imaging using a Zeiss 780 laser scanning microscope. Z-stacks were obtained from the bottom of the gel (marked by blue autofluorescence of the posts) spanning at least 120 μm with a 5.5 μm step size. For quantification of spheroid cell death, the 3D Objects Counter in Fiji (Schindelin et al., Nat Methods. 2012; 9(7):676-82) was used to determine the volume of each object in the red channel (DRAQ7+ nuclei) and green channel (spheroid volume). Custom Fiji scripts were developed to automate file opening, channel splitting, quantification area selection, running the tool, and naming output files while custom R scripts were used to sum the volumes of the 3D objects and generate a summary table of the values. Cell death was quantified as the ratio of DRAQ7 to CtGreen. For quantification of spheroid collective migration, the Plot Z-Axis Profile in Fiji was used to determine the position of peak fluorescence in the green channel. Custom Fiji scripts were developed to automate file opening, running the tool, and naming output files from which the data were extracted and summarized. All scripts are available upon request.

2D vs. 3D gene expression. RCC4 and A498 spheroids were formed overnight at 175 cells/spheroid and resuspended in 2.5 mg/ml collagen at 12 spheroids/μL, injected as 100 μL per well to a 96-well plate, and covered with 200 μL of RCC media. After incubation at 37° C. for 24 hours, 7 wells per replicate were combined for 3 replicates per condition, minced with a blade, and digested with RPMI containing 0.2 mg/ml Liberase-Thermolysin and 100 U/ml DNase I with shaking at 37° C. for 30 minutes. Digests were collected at 845×g for 3 minutes, washed with PBS, and snap-frozen in liquid nitrogen. In parallel, cells were plated in 2D at 5×104 cells/mL as 1 mL per well in a 24-well plate, trypsinized, combined as 6 wells per replicate for 3 replicates per condition, collected, washed, and snap-frozen. RNA was extracted using RNeasy Plus kits (Qiagen, Valencia, CA). For RNA sequencing (RNA-seq), total RNA was submitted to the Genewiz RNA-seq service (Azenta Life Sciences, Burlington, MA). Gene set enrichment analysis (GSEA) was performed using the hallmark collection in the Molecular Signatures Database (Liberzon et al., Cell Syst. 2015; 1(6):417-25).

Statistics. P values were calculated using a two-sided Student's t-test. In RNA-seq, genes were considered to be significantly differentially expressed when log 2 fold change (FC) was ≥1 and the false discovery rate (FDR) was <0.01. In GSEA using clusterProfiler (Yu et al., Omics. 2012; 16(5):284-7), pathways were considered significant when the p value, adjusted for multiple comparisons, was <0.05.

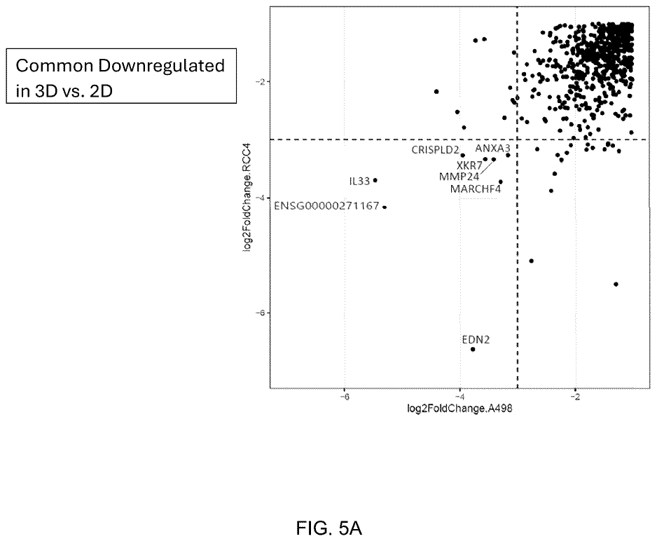

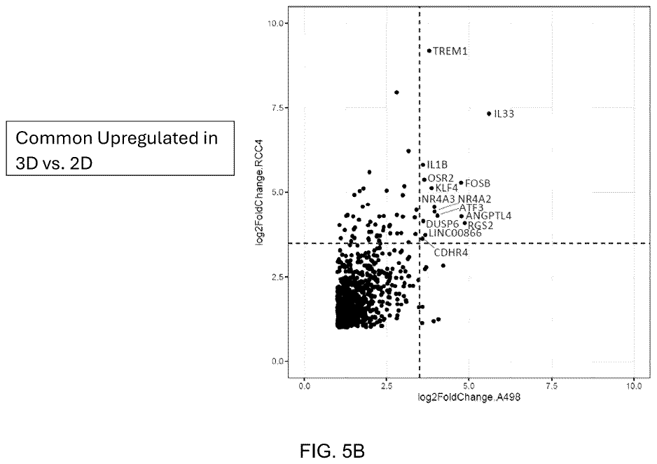

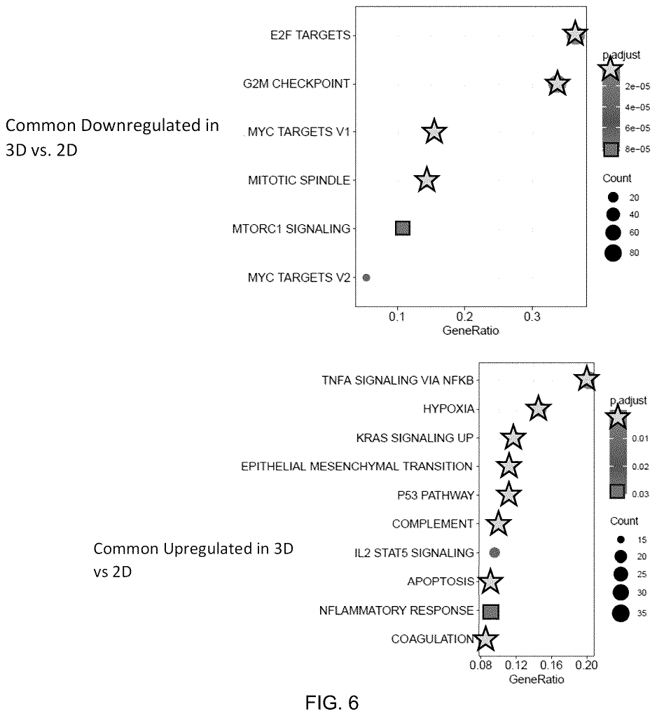

Results. 3D culture induces a primary tumor-like gene expression program in RCC cell lines. A 3D human vascularized RCC-on-a-chip for studies of tumor angiogenesis using the single channel Nortis Bio ParVivo system was previously developed (Miller et al., Neoplasia. 2018; 20(6):610-20). To adapt a more economical microfluidic platform, in this study the idenTx 3 system from Aim Biotech was employed. This chip contains 3 sites that each contain a central matrix region for injecting collagen flanked by 2 parallel media channels for gravity-based flow and administration of therapeutic compounds or cellular reagents such as T cells (FIG. 1A). This design eliminates the need for peristaltic pumps and associated tubing. After preparing the collagen at a desired concentration, a suspension of tumor spheroids of tunable size is injected into the central matrix region for polymerization into a 3D gel that mimics the ECM. Initially, the study focused on the human RCC cell lines A498 and RCC4 due to their widespread use. Selection of these widely available cellular reagents should encourage adoption and assessment of the reproducibility of this platform by other laboratories, which will be an important step to tissues- and organs-on-chips being more widely utilized (Sakolish et al., Sci Rep. 2018; 8(1):14882). Both cell lines represent ccRCC based on their mutant VHL status (Wolf et al., Oncogene. 2020; 39(17):3413-26). Previous data with primary human ccRCC and evidence from a variety of cancer types indicates that the gene expression pattern of tumor cells cultured in a 3D collagen ECM better reflects the gene expression pattern in vivo compared to traditional 2D cultures (Miller et al., Neoplasia. 2018; 20(6):610-20; Brady et al., Prostate. 2020; 80(6):491-9). To test the impact of in vitro culture conditions on the human RCC cell lines, A498 and RCC4 cells as 2D monolayers or as 3D spheroids in collagen were grown and then these cultures were harvested for RNA-seq analysis. Comparing 3D vs. 2D culture of A498 to RCC4 cells, 43.5% of the upregulated and 50.7% of the downregulated genes were differentially expressed in both cell lines (FIG. 1B, FIG. 5A, 5B). GSEA revealed multiple pathways enriched in these overlapping differentially expressed genes (FIG. 6) including hypoxia-inducible genes. Among the hypoxia-inducible genes upregulated in both A498 and RCC4 cells, 12 were also significantly upregulated in primary RCC vs. normal tissues in The Cancer Genome Atlas (TCGA), including 3 proangiogenic genes (VEGFA, ANGPTL4, and PGF) that were previously demonstrated (Miller et al., Neoplasia. 2018; 20(6):610-20) were upregulated in primary RCC vs. normal renal epithelial cell spheroids (FIG. 1C). These results demonstrated that 3D culture of RCC cell lines reproduces a gene expression program that is more similar to in vivo tumors compared to the same cell lines cultured in 2D.

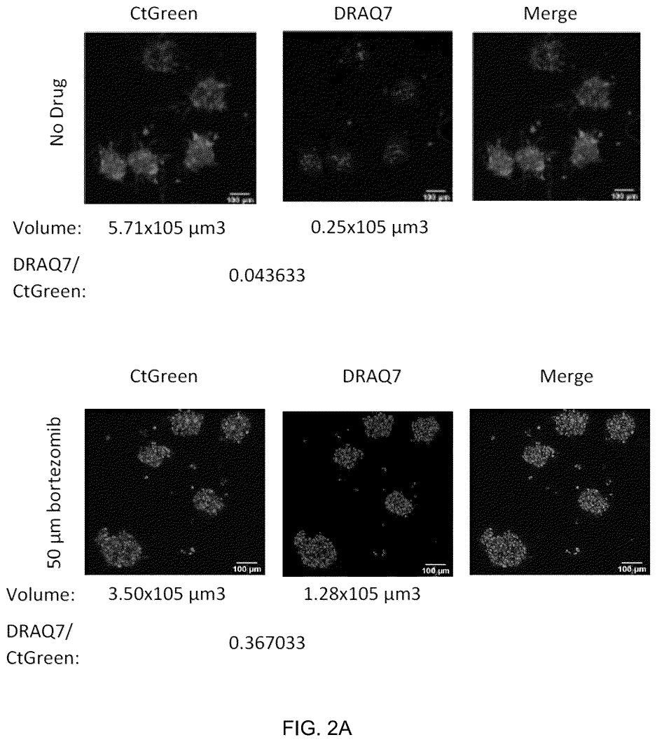

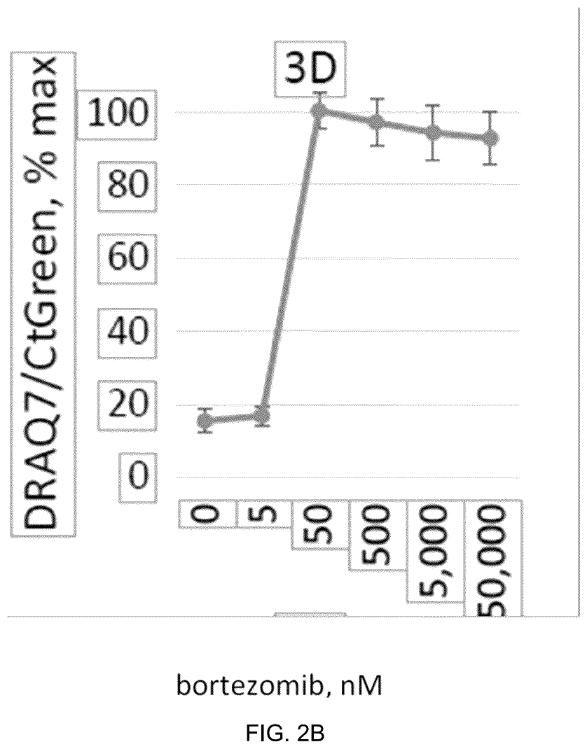

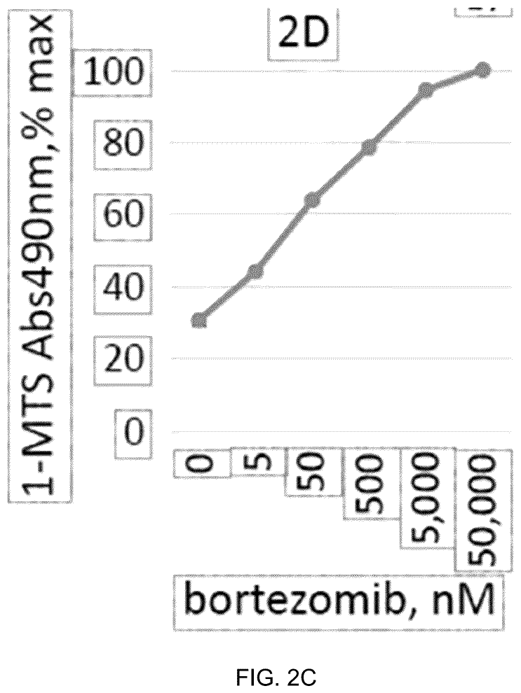

Quantification of cell death in the 3D RCC-on-a-chip. An important aspect of drug testing is the need to quantify cytotoxicity as a function of dose. To implement a cell death readout, spheroids were treated with the proteasome inhibitor bortezomib which stabilizes pro-apoptotic factors. Interestingly, bortezomib was reported to be the tenth most potent cytotoxic drug for A498 cells among a panel of 281 drugs, and 2 kidney cancer cell lines (including A498) were among the top 21 most sensitive cell lines to bortezomib among a panel of 957 cell lines (see, https://www.cancerrxgene.org/). To quantify cell death, the live/dead stain DRAQ7 was included in the media which only fluoresces when incorporated into the nuclei of dead cells. In the absence of bortezomib, spheroid cell death was limited to few cells, mainly in the central region of the spheroids (FIG. 2A, top panels). Administration of bortezomib induced cell death in a dose-dependent fashion, with dead cells observed throughout (FIG. 2A, bottom panels, and FIG. 2B). To quantify cell death, an automated approach was developed for batch processing and unbiased quantification of the ratio of the volume of DRAQ7 staining to total spheroid staining. This demonstrated a sharp inflection of cytotoxicity between 5 and 50 nM of bortezomib, with essentially complete spheroid killing at and beyond 50 nM (FIG. 2B). By contrast, treatment of 2D cultures of A498 performed in parallel, there was a more linear cell death response, which at 50 nM reached only 63.9% of the maximal cell death response and approached saturation at a much higher concentration of 5 μM (FIG. 2C). This reduced 2D cytotoxicity of bortezomib may reflect the continued proliferation of the cells that occurs in 2D monolayers but not in spheroids embedded in a 3D matrix (spheroid size did not increase during the 48-hour experiment). It could also represent an increased cellular sensitivity to proteasomal inhibition when grown in a 3D environment. Consistent with these hypotheses, RNA-seq showed that several pathways associated with cell proliferation were downregulated in 3D while susceptibility to apoptosis was upregulated (FIG. 6). These studies established that the RCC-on-a-chip platform can provide a quantitative readout of cell death in response to a chemotherapeutic agent. They also demonstrated the differential sensitivity of cells grown in 2D vs. 3D conditions.

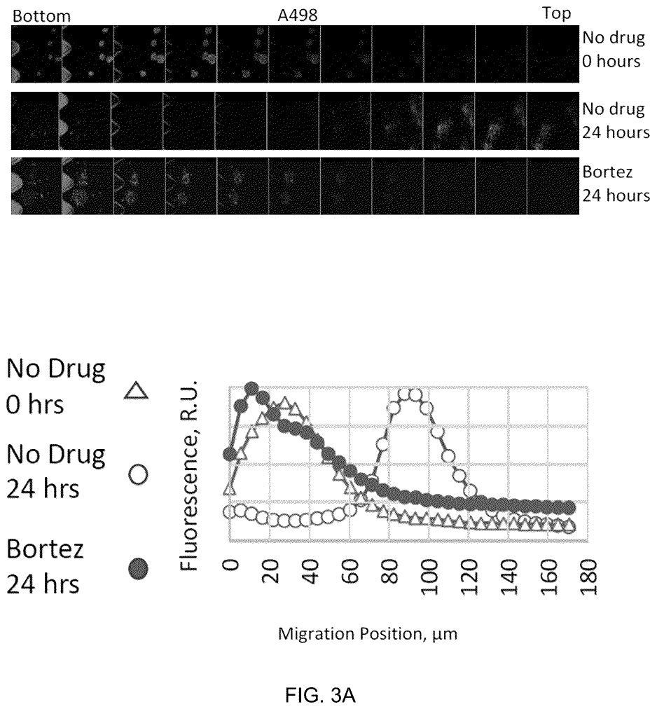

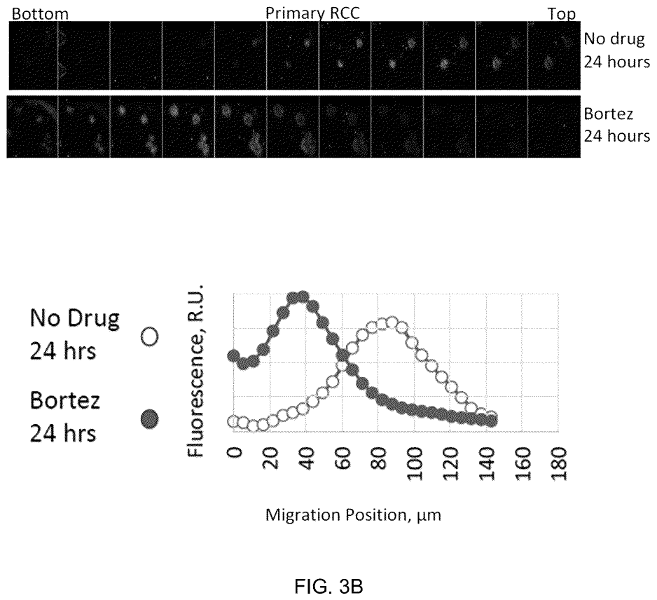

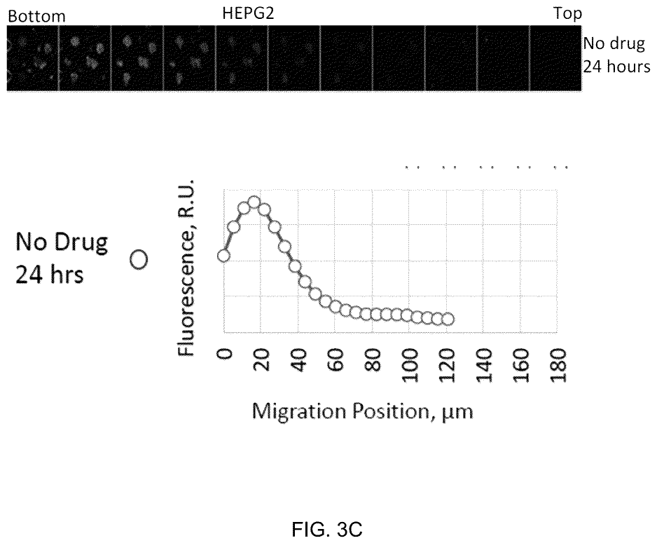

An invasive strand phenotype correlates with spheroid collective migration. Migration and ECM invasion are hallmarks of cancer correlated with metastasis in vivo (Hanahan & Weinberg, Cell. 2011; 144(5):646-74). These processes are difficult to study in 2D culture systems and is labor-intensive, time-prohibitive and expensive in xenograft models. In the cytotoxicity experiments, it was observed that A498 tumor spheroids seeded in the chip exhibited an invasive strand phenotype penetrating from the spheroid mass into the surrounding collagen ECM (FIG. 2A). This phenotype has been reported previously in tumor cell aggregates in collagen (Padmanaban et al., Nature. 2019; 573(7774):439-44; Su et al., Biomaterials. 2021; 275:120922). Interestingly, it was also observed in confocal z-stack montages showing the progression of the images obtained from the bottom through the top of the collagen gel that untreated spheroids were located at a higher position in the gel compared to bortezomib-treated spheroids (FIG. 3A). This was unexpected and suggested that the spheroids were able to collectively invade and migrate as a cohesive mass through the collagen ECM. To test this hypothesis, confocal imaging of A498 spheroids was performed immediately after seeding and after 24 hours of culture. This analysis revealed that whole spheroids were indeed migrating away from the bottom region of the 3D collagen gel, where they had initially collected at the time of introduction to the chip due to gravity (FIG. 3A, upper panel), toward the upper region of the gel (FIG. 3A, middle panel). This collective migration phenotype was not limited to cell lines, as primary ccRCC tumor cell spheroids also exhibited collective migration in the collagen ECM (FIG. 3B). For both A498 and the primary ccRCC spheroids, migration depended on cell viability since it was blocked with a lethal dose of bortezomib. To quantify spheroid migration, the Plot Z-axis profile in Fiji was used to mark the position of the peak of spheroid fluorescence (FIGS. 3A-3C, graphs). This represents an unbiased quantification method that is more efficient than a previously reported method of quantification of the spheroid invasive strand phenotype based on estimating a form factor of individual spheroids (Padmanaban et al., Nature. 2019; 573(7774):439-44). This migratory phenotype was not described in previous tumor-on-a-chip publications with the same microfluidic system (Lee et al., Front Immunol. 2018; 9:416; Pavesi et al., JCI Insight. 2017; 2(12)). To examine the basis for the difference, the hepatocellular HEPG2 cell line that was used in the previous studies was tested and it was observed that this cell line did not exhibit collective migration (FIG. 3C). This suggests that the migratory behavior in 3D culture is a cell-intrinsic property. The RNA-seq data from RCC4 and A498 cells grown in 2D and 3D was interrogated to identify gene expression changes that could explain this behavior. Interestingly, epithelial to mesenchymal transition was among the top gene sets enriched in 3D vs. 2D for both A498 and RCC4 cells (FIG. 6). The acquisition of a hybrid epithelial/mesenchymal phenotype has been shown to promote collective cancer cell migration (Saxena et al., Transl Oncol. 2020; 13(11):100845). Therefore, the RCC-on-a-chip could serve as a tractable platform to study cell-intrinsic migratory behaviors that are enhanced in 3D.

The RCC-on-a-chip identifies compounds that inhibit collective spheroid migration without inducing cell death as a primary mechanism. Given the unique potential to study tumor cell migration in 3D, the feasibility of using the chip for anti-migratory drug screening was tested. First, a panel of migration inhibitors that act through a variety of mechanisms was assembled (Table 1).

TABLE 1
Antimigratory compounds

| Compound | Target | Concentrations |
|---|---|---|
| saracatinib | SRC | 5 and 50 μM (48) |
| defactinib | FAK | 5 and 50 μM (49) |
| latrunculin A | G-actin | 0.5 and 5 μM (50) |
| blebbistatin | Myosin II | 5 and 50 μM (51) |
| RKI-1447 | ROCK1/2 | 5 and 50 μM (52) |
| AT13148 | ROCK1/2 AKT1/2/3 | 5 and 50 μM (53) |
| ISO-1 | MIF | 50 and 500 μM (54) |

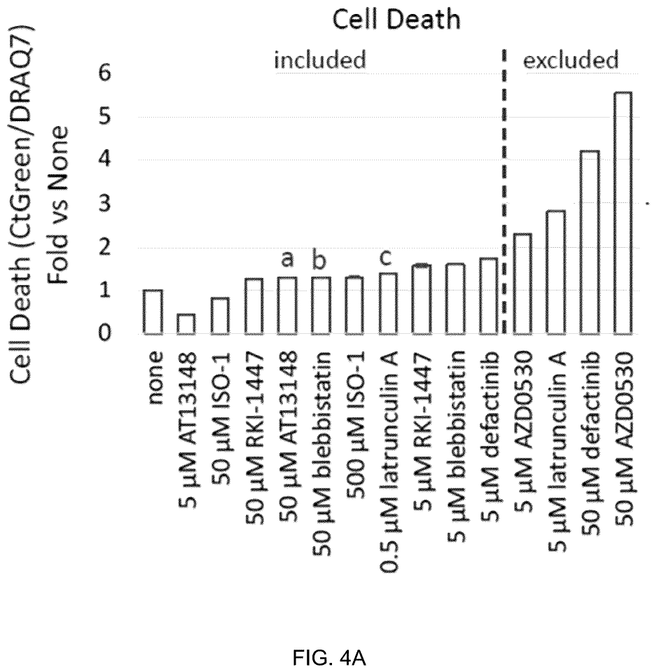

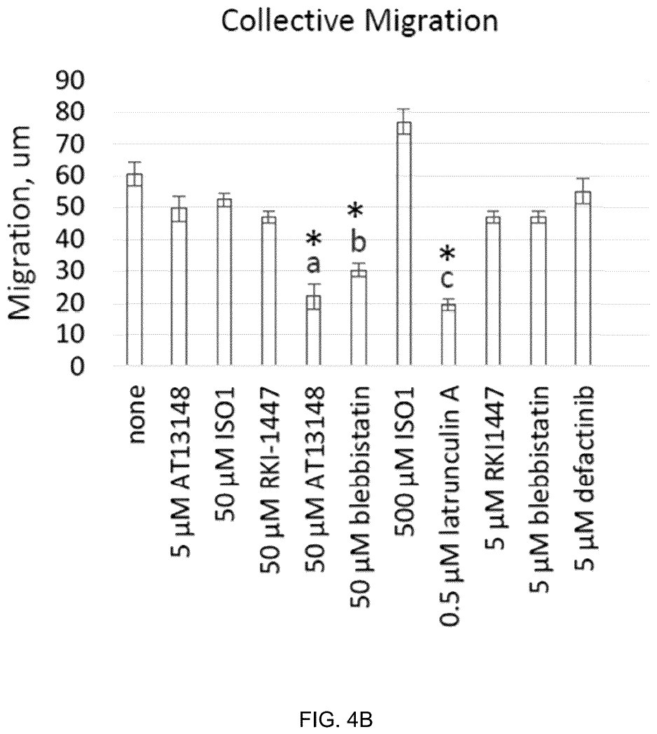

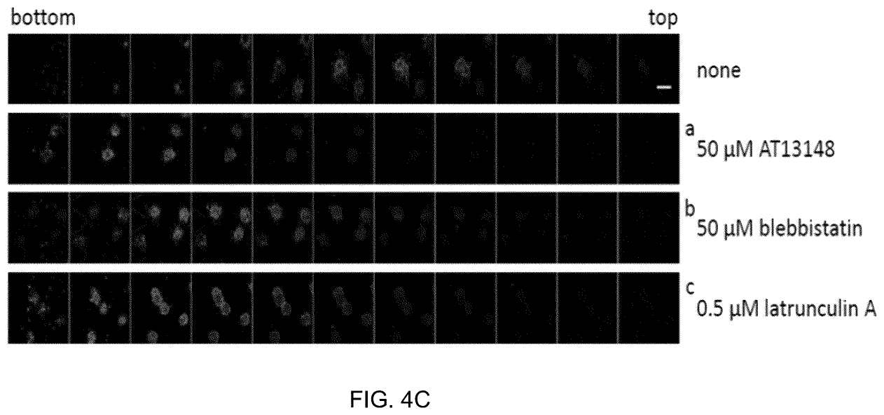

Each compound was initially screened at 2 concentrations based on prior reports. In the simplest scenario, spheroid migration can be inhibited by increased cytotoxicity. However, since image analysis scripts enabled simultaneous automated quantification of spheroid cell death and collective migration, the experiment allowed for the identification of compounds that could inhibit migration without killing the cells. This analysis revealed that while 0.5 μM latrunculin A, 50 μM blebbistatin, and 50 μM AT13148 did not significantly increase cell death (FIG. 4A), they significantly inhibited collective spheroid migration by 70, 52, and 65%, respectively (FIGS. 4B, 4C). These experiments revealed that the direct anti-migratory effects of drugs can be uncoupled and studied separately from their cytotoxic effects in the RCC-on-a-chip.

Discussion. In this study, the adaptation of microfluidic systems from a previously reported human vascularized RCC-on-chip for studies of tumor angiogenesis (Miller et al., Neoplasia. 2018; 20(6):610-20) was expanded to a more economical platform to assess novel therapeutic reagents targeting RCC tumor cells including apoptosis-inducing compounds, antimigratory compounds, and tumor-antigen redirected CD8+ T cells. The commercial idenTx 3 microfluidic platform was selected to facilitate the ease of adoption by standard tissue cultures laboratories that do not have microfabrication capabilities. While this approach lacks the prior-reported capability of constructing a vascular lumen crossing a tumor spheroid-laden ECM, key advantages include not only markedly reduced cost but also ease of use with gravity-driven flow and no required peripheral components such as a syringe pump or perfusion system. Here, the chip assay system has been optimized to simultaneously observe and quantify spheroid cell death and collective migration.

The gene expression profiling studies identified significant changes in cells cultured in 3D vs. 2D, and cells cultured in 3D had an expression profile closer to primary tumors. The phenotypic consequences of this more physiologic expression profile in the RCC-on-a-chip were explored in a variety of assays. For example, cytotoxicity from the apoptosis-inducing agent bortezomib was observed at lower concentration in 3D vs 2D cultures. Many reports in the literature have documented increased resistance of 3D spheroids compared to 2D monolayers for drugs that rely on the generation of reactive oxygen species or inhibition of proliferation owing to the lack of spheroid penetration or reduced effectiveness of drugs in the hypoxic and senescent central region of tumor spheroids (Tosca et al., Biomedicines. 2023; 11(4); Däster et al., Oncotarget. 2017; 8(1):1725-36; Imamura et al., Oncol Rep. 2015; 33(4):1837-43). Conversely, increased drug sensitivity of 3D spheroids compared to 2D monolayers has been shown for other drugs such as EGFR inhibitors and kinase inhibitors (Sirenko et al., Assay Drug Dev Technol. 2015; 13(7):402-14; Kaur et al., Cancer Treat Res Commun. 2021; 29:100463; Vinci et al., BMC Biol. 2012; 10:29; Patra et al., PLoS One. 2020; 15(12):e0244549), suggesting that for certain antitumor mechanisms, 3D spheroid culture may be a more sensitive platform.

Another phenotype that was discovered in the 3D RCC-on-a-chip was collective migration. It was found that spheroid collective migration can be adapted to screen and evaluate anti-migratory compounds. Importantly, not only single-cell but also collective migration of tumor cells at the tumor ECM interface in vivo, with the latter being less common but more efficient, are alternate steps on the path to metastasis (Clark & Vignjevic, Curr Opin Cell Biol. 2015; 36:13-22; Nagai et al., J Biochem. 2020; 167(4):347-55). Key advantages of the current assay compared to previously utilized methods include 1) the ability to simultaneously monitor 3D collective cell migration and cell death in the same assay, which excludes compounds that simply kill cells as the main mechanism of inhibiting migration; 2) inclusion of a biomimetic collagen ECM which confers invasive properties to the spheroids; 3) script-based, automated, unbiased quantification of the collective migration, and 4) commercially available and affordable microfluidic tissue culture chips. Regarding the latter, the idenTx chip is newly available in a 48-site plate format, allowing scaling to high-throughput screening.

In summary, the RCC-on-a-chip provides an ideal and physiologically relevant testing platform given its low cost, quantitative rigor, and ability to simultaneously measure both viability and migration. The chip-based approach focuses on collective cancer cell migration, is compatible with cell lines or primary cultures of tumor cells, and could be used in conjunction with other human microphysiological systems for toxicity assessment incorporating normal renal tubule (Weber et al., Kidney Int. 2016; 90(3):627-37) and normal liver (Ewart et al., Commun Med (Lond). 2022; 2(1):154) to prioritize therapeutics with a higher likelihood of success in clinical trials.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1-71. (canceled)

72. A method of screening a test compound for anti-metastatic properties, the method comprising:
adding a tumor spheroid-collagen solution to a central matrix channel of a microfluidic culture chip;
polymerizing biomimetic collagen within the tumor spheroid-collagen solution to form a biomimetic collagen matrix;
adding a first amount of a test compound media to an upper injection port of the microfluidic culture chip;
adding a second amount of the test compound media to a lower injection port of the microfluidic culture chip wherein the second amount is less than the first amount;
staining the microfluidic culture chip with a fluorescence compound, wherein the staining creates labeled tumor spheroids; and
performing Z-stack confocal imaging using a laser scanning microscope to form Z-stacks and to assess anti-gravitational migration of the labeled tumor spheroids within the collagen matrix thereby screening the test compound for anti-metastatic properties.

73. The method of claim 72, further comprising adding tumor spheroids into a collagen solution to form the tumor spheroid-collagen solution.

74. The method of claim 72, wherein the tumor spheroids are A498 spheroids or HEPG2 spheroids.

75. The method of claim 72, wherein the tumor spheroids have 100-500 cells/spheroid.

76. The method of claim 72, comprising adding 1 mg/mL or 2.5 mg/mL tumor spheroid-collagen solution to the microfluidic culture chip.

77. The method of claim 72, wherein polymerizing collagen within the tumor spheroid-collagen solution is for 20-40 minutes at 37° C.

78. The method of claim 72, comprising adding 50-100 μL of the test compound media in the upper injection port and 25-75 μL of the test compound media in the lower injection port.

79. The method of claim 72, wherein the test compound is a putative anti-migratory compound.

80. The method of claim 72, further comprising creating a Montage gallery of each z-stack position of labeled tumor spheroids.

81. The method of claim 72, wherein the Z-stacks are obtained from a bottom of the collagen matrix that is marked by fluorescence of one or more posts separating the central matrix channel and parallel media channels.

82. The method of claim 72, wherein the z-stacks span a 4-8 μm step size.

83. The method of claim 72, wherein an unbiased script-based automated system quantifies at least one of a cell invasion, a cell migration, or a cell death.

84. A system for screening a compound for anti-metastatic properties against renal cell carcinoma (RCC), the system comprising:
a microfluidic culture chip, wherein the microfluidic culture chip comprises at least 2 sites per chip, each site containing injection ports for a central matrix channel and parallel media channels flanking the sites, and posts with gaps in between on each side of the matrix channel separating the central matrix channel and parallel media channels;
a biomimetic collagen-RCC tumor spheroid matrix in the central matrix channel of the microfluidic culture chip, wherein collagen within the collagen-RCC tumor spheroid matrix is polymerized and comprises 4-20 tumor spheroids/μL; and
an upper injection port and a lower injection port, each comprising a test compound media with a pressure gradient between the upper injection port and the lower injection port.

85. The system of claim 84, wherein tumor spheroids within the collagen-RCC tumor spheroid matrix are A498 spheroids.

86. The system of claim 84, wherein tumor spheroids within the collagen-RCC tumor spheroid matrix have 100-500 cells/spheroid.

87. The system of claim 84, wherein collagen within the collagen-RCC tumor spheroid matrix was polymerized at 37° C. for 20-40 minutes.

88. The system of claim 84, comprising 50-100 μL of the test compound media in the upper injection port and 25-75 μL of the test compound media in the lower injection port.

89. The system of claim 84, wherein a test compound within the test compound media is a putative anti-migratory compound.

90. The system of claim 84, wherein the bottom of the collagen-RCC tumor spheroid matrix is marked by fluorescence of one or more posts separating the central matrix channel and parallel media channels.

91. The system of claim 84, comprising a processor that executes an unbiased script-based automated quantification at least one of a cell invasion, a cell migration, or a cell death.
